(19) United States
(12) Patent Application Publication
(10) Pub. No.: US 2011/0230906 A1
(43) Pub. Date: Sep. 22, 2011

(54) DEVICES, METHODS AND KITS FOR FORMING TRACTS IN TISSUE

(75) Inventors: D. Bruce Modesitt, San Carlos, CA (US); George D. Hermann, Portola Valley, CA (US); Michael Drews, Palo Alto, CA (US); Joseph F. Paraschac, Campbell, CA (US); Brian Andrew Ellingwood, Sunnyvale, CA (US); Dan J. Hammersmark, San Mateo, CA (US)

(73) Assignee: Arstasis, Inc., San Carlos, CA (US)

(21) Appl. No.: 13/004,848

(22) Filed: Jan. 11, 2011

Related U.S. Application Data
(60) Provisional application No. 61/294,103, filed on Jan. 11, 2010.

(51) Int. Cl. A61B 17/34

(52) U.S. Cl. 606/185

(57) ABSTRACT

Described here are methods, devices and kits for locating tissue and/or forming one or more tracts in tissue. In some variations, tissue may be located (e.g., using one or more optical sensors, ultrasound sensors, thermal sensors, or the like) and one or more tracts may be formed through the tissue after it has been located. In certain variations, the same device may be used both to locate tissue and to form one or more tracts in the tissue. In some variations, a tissue-piercing member for forming one or more tracts in tissue may comprise a first elongated portion and a second elongated portion, and an angle therebetween.

### RELATED APPLICATION DATA

The present application claims the benefit under 35 U.S.C. § 119 to U.S. Provisional Application Ser. No. 61/294,103, filed Jan. 11, 2010. The foregoing application is hereby incorporated by reference into the present application in its entirety.

### FIELD

In general, the methods, devices and kits described herein are useful for procedures involving body tissue. More specifically, the methods, devices and kits described herein are useful for locating target tissue and/or for forming one or more tracts in tissue.

### BACKGROUND

A number of devices and methods have previously been described for forming tracts in or through tissue. For example, U.S. patent application Ser. Nos. 10/844,247 (published as US 2005/0267520 A1), Ser. No. 10/888,682 (published as US 2006/0009802 A1), Ser. No. 11/432,982 (published as US 2006/0271078 A1), Ser. No. 11/544,149 (published as US 2007/0032802 A1), Ser. No. 11/544,177 (published as US 2007/0027454 A1), Ser. No. 11/544,196 (published as US 2007/0027455 A1), Ser. No. 11/544,317 (published as US 2007/0106246 A1), Ser. No. 11/544,365 (published as US 2007/0032803 A1), Ser. No. 11/545,272 (published as US 2007/0032804 A1), Ser. No. 11/788,509 (published as US 2007/0255313 A1), Ser. No. 11/873,957 (published as US 2009/0105744 A1), Ser. No. 12/507,038 (filed on Jul. 21, 2009), and 12/507,043 (filed on Jul. 21, 2009), all of which are incorporated herein by reference in their entirety, describe devices and methods for forming tracts in tissue. In general, the tracts described there may self-seal or seal after they have been formed, with minimal or no need for supplemental closure devices or techniques. These tracts may be quite useful in providing access to a tissue location (e.g., an organ lumen) so that one or more tools may be advanced through the tract, and a procedure may be performed. Given the tremendous applicability of such methods, additional devices and methods of forming tracts in tissue would be desirable. It would also be desirable to easily and accurately locate tissue (e.g., prior to tract formation).

### SUMMARY

Described here are methods, devices and kits for locating or identifying tissue, and/or for forming one or more tracts in tissue. In some variations, the devices may comprise one or more optical components, tactile components, audio components, and the like, such as one or more sensors and/or cameras. The components may be positioned at any appropriate location on and/or in the devices, and may be internal to the body or external to the body during use. The components may be useful, for example, in locating and/or identifying target tissue. As an example, devices and methods described here may be helpful in locating tissue that might otherwise be difficult to locate (e.g., locating a target vessel in an obese person). They may also be useful in positioning a tissue-piercing member at a particular angle with respect to target tissue, in order to form a desired tract through the target tissue. Moreover, devices and methods described here may be used to direct a tissue-piercing member along a desired path through tissue, after the tissue-piercing member has already punctured the skin surface to get to the tissue.

In certain variations, device and/or methods described here may be used to form a self-sealing tissue tract. A self-sealing tissue tract does not need interventional devices or methods to help it seal—by definition, it seals by itself. For example, a self-sealing tissue tract does not need a plug, energy, sealants, clips, sutures, or the like to help it seal. Rather, a self-sealing tissue tract may seal when opposing tissue portions along the tract contact each other and form a seal. This may occur, for example, when the angle of the tract relative to the tissue wall is relatively shallow, which may result in the tract having a relatively long length and/or high surface area. Blood pressure may cause the tissue portions to come into contact with each other and natural clotting factors and the like may cause them to form a seal. Of course, it should be understood that, as described later herein, manual pressure or compression may be applied to a self-sealing tract to expedite its sealing, without affecting the self-sealing nature of the tract.

In some variations, a device may comprise one or more sensors and a method may comprise sensing at least one useful parameter, such as temperature, pressure, tissue identification or location (e.g., nerves or various anatomical structures), blood flow within a vessel, and combinations thereof. For example, in certain variations, the parameter may be blood flow within a vessel, and the method may further comprise repositioning the device if blood flow within a vessel is detected. In some variations, an output display, such as a monitor, may be provided to allow a user to easily view the surroundings of a device during a tissue-locating procedure. Such an output display may also be used when one or more tracts are being formed through tissue. An operator may view the output display and may adjust the device or devices accordingly.

In certain variations, a device may pass through tissue once to form a single tract in the tissue. The formation of a single tissue tract may, for example, allow for a relatively easy recovery period after one or more desired procedures have been performed through the tissue tract. One embodiment is directed to a system for forming a tract in a targeted tissue structure wall located across a thickness of tissue from a point of patient access, comprising a tissue-piercing member comprising a proximal elongated portion and a distal elongated portion coupled to the proximal elongated portion, the distal portion comprising a tissue-piercing tip; and a mandrel; wherein a lumen is formed through both portions of the tissue-piercing member and configured to slidably receive the mandrel, such that when the mandrel is received by both elongated portions, the elongated portions assume a first orientation relative to each other, and when the mandrel is withdrawn proximally out of at least the distal elongated portion, the elongated portions assume a second orientation relative to each other. The system may further comprise a guidewire slideably coupled through the lumen of the tissue-piercing member and advanced from the point of patient access across at least a portion of the targeted tissue structure wall. The proximal and distal elongated portions of the tissue-piercing member may be coupled with a bending section. The bending section may assume a predetermined bent configuration when unloaded. The predetermined bent configuration may be selected to place proximal and distal elongate members in the second orientation relative to each other when coupled with the bending section and not restrained by the mandrel. The proximal and distal elongated portions of the tissue-piercing member may be coupled with a joint. The system may further comprise a biasing member coupled to the joint and configured to bias the joint to rotate to a predetermined configuration when unloaded. The predetermined configuration may be selected to place proximal and distal elongate members in the second orientation relative to each other when coupled with the joint and not restrained by the mandrel. At least one of the first and second elongated portions may be substantially straight when unloaded. At least one of the first and second elongated portions may have a bent configuration when unloaded. The system may further comprise an elongated deployment member movably coupled to the tissue-piercing member and configured to be manipulated by an operator to apply loads to the tissue-piercing member. The elongated deployment member may define a deployment lumen configured to accommodate slidable coupling of the tissue-piercing member with the elongated deployment member. The tissue-piercing member may be biased to assume the second configuration when unloaded, and the mandrel may comprise a structural stiffness selected to maintain the tissue-piercing member in the first configuration when inserted through both the proximal and distal elongated portions. The tissue-piercing member may be biased to assume the second configuration when unloaded, and the mandrel may comprise a structural stiffness selected to urge the tissue-piercing member back into the first configuration after the second configuration has been assumed, such reconfiguration being accomplished by applying insertional forces on the mandrel relative to the tissue-piercing member to insert the mandrel back through at least a portion of the lumen defined through the distal elongate portion of the tissue-piercing member. The tissue-piercing member may be a needle. The tissue-piercing member may comprise at least one shape-memory material. The tissue-piercing member may comprise at least one super-elastic material. The super-elastic material may comprise nitinol. An articulation angle may be defined between a longitudinal axis of the proximal tissue-piercing member portion and a longitudinal axis of the distal tissue-piercing member portions, and the articulation angle with the proximal and distal elongated portions in the first orientation may be between about 135 degrees and about 180 degrees. The articulation angle with the proximal and distal elongated portions in the first orientation may be about 175 degrees. An articulation angle may be defined between a longitudinal axis of the proximal tissue-piercing member portion and a longitudinal axis of the distal tissue-piercing member portions, and the articulation angle with the proximal and distal elongated portions in the second orientation may be between about 90 degrees and about 135 degrees. The articulation angle with the proximal and distal elongated portions in the second orientation may be about 100 degrees. The tissue-piercing member may be configured to be advanced through the thickness of tissue with the tissue-piercing tip at a first orientation angle relative to the targeted tissue structure wall until the tissue-piercing tip is located adjacent the targeted tissue structure wall, after which the mandrel may be at least partially withdrawn relative to the tissue-piercing member to cause the tissue-piercing member to assume the second orientation and place the tissue-piercing tip at a second orientation angle relative to the targeted tissue structure wall that is less than the first orientation angle relative to the targeted tissue structure wall, the second orientation angle being selected to cause the tissue-piercing tip to be advanceable into the targeted tissue structure wall with a trajectory configured to leave behind a tract through the targeted tissue structure wall that is self-sealing after the tissue-piercing member has been withdrawn. The first orientation angle of the tissue-piercing tip relative to the targeted tissue structure wall may be between about 30 degrees and about 60 degrees. The first orientation angle of the tissue-piercing tip relative to the targeted tissue structure wall may be about 45 degrees. The second orientation angle of the tissue-piercing tip relative to the targeted tissue structure wall may be between about 2 degrees and about 30 degrees. The second orientation angle of the tissue-piercing tip relative to the targeted tissue structure wall may be about 10 degrees.

In some variations, a device may comprise at least one tissue-piercing member. In certain variations, the tissue-piercing member may be a needle. The needle may be hollow or solid, and may have any suitable tip. That is, the tip may have any suitable shape (conical, offset conical, etc.), may be blunt, sharpened or pointed, and may be beveled or non-beveled. Other appropriate tissue-piercing member configurations may also be used. In some variations, a tissue-piercing member may comprise at least one shape-memory material and/or at least one super-elastic material. Such materials may, for example, allow the tissue-piercing member to assume different configurations under different conditions.

In certain variations, a system for forming a tract in tissue may comprise a mandrel and a tissue-piercing member comprising a first elongated portion and a second elongated portion integral with the first elongated portion and comprising a tissue-piercing tip. The tissue-piercing member may have a lumen configured to receive the mandrel. Additionally, the tissue-piercing member may have a first configuration in which the first and second elongated portions have a first angle therebetween, and a second configuration in which the first and second elongated portions have a second angle therebetween that is different from the first angle. The first angle may be from about 135° to about 180° (e.g., about 175°), and/or the second angle may be from about 90° to about 135° (e.g., about 100°). The tissue-piercing member may be in the first configuration when the mandrel is disposed within the lumen.

In certain variations, a system for forming an oblique tract in an arterial wall may comprise a tissue-piercing member comprising a first elongated portion and a second elongated portion integral with the first elongated portion and comprising a tissue-piercing tip. The tissue-piercing member may have a lumen. Additionally, the first and second elongated portions may have an angle therebetween that is from about 120° to about 180° (e.g., from about 135° to about 180°, such as about) 175°.

The systems may further comprise an elongated member. The tissue-piercing member may be coupled to the elongated member and configured to be deployed therefrom. The tissue-piercing member may be slidably disposed within the elongated member.

Methods for forming tracts in tissue are also described here. In accordance with some methods, a device may be used to locate or identify tissue. The device may, for example, be one of the devices described herein. The methods may include determining the location of tissue and/or the location of a device with respect to the tissue. The tissue may be visualized and/or identified with ultrasonography (e.g., Doppler ultrasonography), thermal sensing, optical sensing, and the like. In some cases, a tract may be formed in tissue and one or more tools may be advanced through the tissue tract. In certain variations one or more procedures may be performed adjacent to, through, or on the tissue.

In some variations, a method for forming a tract in a tissue wall (e.g., a vessel wall, such as an artery wall) may comprise heating or cooling a device to a temperature that is different from 37° C., advancing a device through tissue while measuring the temperature of a portion of the device, and advancing a tissue-piercing member through the tissue when the temperature of the portion of the device changes and thereby indicates that the device is in the proximity of a tissue wall, so that the tissue-piercing member is advanced through the tissue wall, where advancing the tissue-piercing member through the tissue wall forms a tract in the tissue wall. In certain variations, the portion of the device may comprise a portion of the tissue-piercing member. The device may be heated or cooled as the device is advanced through the tissue, and/or prior to advancement through the tissue. The device may comprise a heater element that heats the device. The method may comprise measuring the temperature of a plurality of different portions of the device.

In certain variations, a method for forming a tract in tissue may use a tissue-piercing member comprising a first elongated portion and a second elongated portion integral with the first elongated portion. The method may comprise advancing the tissue-piercing member (e.g., over a guide member, such as a guidewire) through a portion of tissue, where the first and second elongated portions have an angle therebetween that is from about 120° to about 180° (e.g., from about 135° to about 180°, such as about) 175°. The first angle may, for example, be from about 135° to about 180° (e.g., about) 175°, and/or the second angle may, for example, be from about 90° to about 135° (e.g., about 100°).

In certain variations, a method for forming a tract in a tissue wall may comprise displacing a portion of subcutaneous tissue to from a space adjacent the tissue wall, advancing at least a portion of a tissue-piercing member into the space, the tissue-piercing member comprising a first elongated portion and a second elongated portion integral with the first elongated portion, the first and second elongated portions having an angle of about 90° to about 180° therebetween, and advancing the second elongated portion into the tissue wall to form a tract in the tissue wall. Displacing the portion of subcutaneous tissue may comprise removing the portion of subcutaneous tissue. For example, the portion of subcutaneous tissue may be dissected (e.g., using a tissue dissector). In some variations, the portion of subcutaneous tissue may be bluntly dissected. The angle between the first and second elongated portions may, for example, be from about 90° to about 135° (e.g., about 90° or about) 100°. Alternatively or additionally, the angle between the first and second elongated portions may, for example, be from about 120° to about 180°.

In certain variations, a method for forming a tract in a tissue wall may comprise positioning an elongated member adjacent an external surface of the tissue wall, applying a force (e.g., a pushing force) to the external surface of the tissue wall with the elongated member to position the tissue wall with the elongated member, and advancing a tissue-piercing member through the tissue wall while the tissue wall is positioned by the elongated member, to form a tract in the tissue wall. In certain variations, the tissue-piercing member may be advanced along a surface of the elongated member and into the tissue wall. In some variations, the elongated member may comprise a lumen therethrough, and the tissue-piercing member may be advanced through the lumen of the elongated member and into the tissue wall. The tissue wall may be positioned without contacting an internal surface of the tissue wall, and/or without capturing an external surface of the tissue wall.

In certain variations, a method for forming a tract in a tissue wall may comprise positioning an elongated member adjacent an external surface of the tissue wall, tensioning the external surface of the tissue wall with at least one tensioning member, and advancing a tissue-piercing member through the tissue wall while the tissue wall is tensioned by the at least one tensioning member, to form a tract in the tissue wall.

The tissue or portion thereof may be tissue of a vessel wall, such as tissue of an arterial wall. The tissue or portion thereof may be tissue of an organ. The organ may be selected from the group consisting of an organ of the cardiovascular system, an organ of the digestive system, an organ of the respiratory system, an organ of the excretory system, an organ of the reproductive system, and an organ of the nervous system. The organ may be an artery or a stomach.

A tissue-piercing member may enter the tissue at a first location, and exit the tissue at a second location, and the length between the first and second locations may be greater than the thickness of the tissue. A tract may be formed in the tissue, and the length of the tract may be greater than the thickness of the tissue.

A method may further comprise advancing one or more closure devices and/or tools into (e.g., through) a tract, and/or withdrawing a tissue-piercing member from tissue. In some variations, a tract may self-seal after a tissue-piercing member that was used to form the tract has been withdrawn from tissue. As described above, a self-sealing tissue tract does not need interventional devices or methods to help it seal—by definition, it seals by itself. For example, a self-sealing tissue tract does not need a plug, energy, sealants, clips, sutures, or the like to help it seal. The tract may self-seal within 15 minutes or less (e.g., within 12 minutes or less, within 10 minutes or less, within 5 minutes or less, within 3 minutes or less, within 1 minute or less). Of course, one or more closure devices or methods may also be used to seal the tract. Some variations of methods may comprise rotating a tissue-piercing member while the tissue-piercing member is advanced through tissue.

A tract may form an angle of less than or equal to about 30° (e.g., less than or equal to about 15°, less than or equal to about 10°, less than or equal to about 5°, about 1° to about 30°, about 1° to about 19°, about 1° to about 15°, about 1° to about 10°, about 1° to about 5°, about 5° to about 15°, about 5° to about) 10° with respect to a longitudinal axis of a tissue wall in which the tract is formed, or a surface of tissue in which the tract is formed.

The methods described here may also comprise delivering one or more fluids or agents to the tissue. The fluids may be useful, for example, for irrigation, sterilization, treatment of tissue (therapeutic, etc.), or the like. The fluids may comprise any suitable agent or combination of agents. For example, the agent may be selected from the group consisting of antibiotics, antiseptics, sterilizing agents, chemotherapeutics, non-steroidal anti-inflammatory drugs (NSAIDs), cyclooxygenase-1 (COX-1) inhibitors, cyclooxygenase-2 (COX-2) inhibitors, opioids, or any other drug or agent, and mixtures and combinations thereof

Some variations of methods described here may be used to form a single tract in tissue, or may be used to form one or more tracts in tissue by advancing a single tissue-piercing member into the tissue. This may, for example, result in minimal stress on the tissue, and/or may reduce the likelihood of damage or harm to the tissue. Moreover, the tissue may recover relatively quickly, thereby resulting in relatively short procedure time.

Kits incorporating one or more of the devices described here, in conjunction with one or more tools, instructions for use, etc., are also described here.

### BRIEF DESCRIPTION OF THE DRAWINGS

FIGS. 1A-1E depict variations of a device and method for forming a tract in tissue.

FIG. 2 depicts variations of a device and method that use a scope to locate tissue.

FIG. 3 is an illustrative depiction of a variation of a self-sealing tract through a vessel wall.

FIG. 4 depicts variations of a device and method that use an audio sensor to locate tissue.

FIGS. 5A and 5B depict additional variations of devices and methods that use audio sensors to locate tissue.

FIGS. 6A-6D depict variations of devices and methods that use ultrasound sensors to locate tissue.

FIG. 7 depicts variations of a device and method that use external ultrasound to locate tissue.

FIGS. 8A and 8B depict variations of devices and methods that use ultrasound probes transdermally to locate tissue.

FIG. 9A depicts variations of a device and method that use electrical impedance measurements to locate tissue, and FIG. 9B is an illustrative variation of the device of FIG. 9A.

FIGS. 10A and 10B depict variations of a device and method for locating tissue using thermal sensing and for forming a tract in the tissue.

FIG. 10C shows a variation of a device that uses thermal sensing to locate tissue.

FIGS. 11A and 11B depict variations of a device and method for positioning tissue to from a tract through the tissue.

FIGS. 12A-12E depict variations of a device and method for tensioning tissue and forming a tract through the tensioned tissue.

FIGS. 13A-13J depict variations of a device and method for forming a tract in tissue.

FIGS. 14A-14G depict additional variations of a device and method for forming a tract in tissue.

FIGS. 15A-15H depict an illustrative method for forming a tract in stomach tissue.

FIGS. 16A-16D depict an illustrative method of accessing the pericardial space.

FIGS. 17A-17K depict an illustrative method for forming a tract in heart tissue.

### DETAILED DESCRIPTION

Described here are methods, devices and kits for locating or identifying tissue, and/or for forming one or more tracts in tissue. In some cases, a device may be configured both to locate tissue and to form one or more tracts in the tissue. When tissue is located or identified prior to tract formation, the result may be relatively controlled and/or predictable tract formation, with a low likelihood of error. As a result, the overall outcome of a procedure may be improved. For example, a tissue-piercing member may be unlikely to be advanced through an untargeted tissue location (e.g., through the side of a vessel, in cases in which it is preferred for the tissue-piercing member to go directly into the main lumen of the vessel). Depending on the nature of the procedure at hand, there may also be a lower incidence of tissue-piercing members being stuck too close to vessel branches (e.g., the femoral bifurcation) or too high relative to the femoral head (which may, for example, result in a retroperitoneal bleed). In applicable procedures, there may be a lower likelihood of a tissue-piercing member inadvertently being advanced through the inguinal ligament on the way to a vessel. As a result, the operator may be able to avoid having to pass one or more devices through the inguinal ligament during a procedure. Devices, methods and kits described here may also be associated with a relatively low morbidity, at least for the reasons described above.

Devices, methods and kits described here may be used in any appropriate tissue, such as a target vessel or a tissue region within a larger tissue body (e.g., a leg muscle, groin, arm, etc.). The larger tissue body may, for example, comprise dermal tissue, connective tissue (e.g., adipose tissue or fat), muscle, etc. The tissue may be tissue of the cardiovascular system, the digestive system, the respiratory system, the excretory system, the reproductive system, the nervous system, or the like. Additionally, some variations of devices, methods and kits described here may be used to reliably and accurately orient one or more tissue-piercing members relative to tissue for tract formation through the tissue. For example, a needle may be oriented relative to a longitudinal axis of a vessel, in order to achieve a desirable elongated angled path through the vessel wall during tissue tract formation.

In certain variations, a device described here may be used to form a single tract through tissue without having to form any other tracts through the tissue when used. By minimizing the total number of tissue tracts formed in a procedure, such a device may also result in a reduced likelihood of excessive bleeding from the tissue tract, as well as a relatively quick recovery time.

Devices described here may take on a variety of forms and may have a number of additional or useful features, as will be described in detail below.

In some cases, when the devices described here are used to form tracts in or through tissue, the tracts may be capable of self-sealing with minimal or no additional sealing efforts, as described, for example, in U.S. patent application Ser. Nos. 10/844,247 (published as US 2005/0267520 A1), Ser. No. 10/888,682 (published as US 2006/0009802 A1), Ser. No. 11/432,982 (published as US 2006/0271078 A1), Ser. No. 11/544,149 (published as US 2007/0032802 A1), Ser. No. 11/544,177 (published as US 2007/0027454 A1), Ser. No. 11/544,196 (published as US 2007/0027455 A1), Ser. No. 11/544,317 (published as US 2007/0106246 A1), Ser. No. 11/544,365 (published as US 2007/0032803 A1), Ser. No. 11/545,272 (published as US 2007/0032804 A1), Ser. No. 11/788,509 (published as US 2007/0255313 A1), Ser. No. 11/873,957 (published as US 2009/0105744 A1), Ser. No. 12/467,251 (filed on May 15, 2009), Ser. No. 12/507,038 (filed on Jul. 21, 2009), and Ser. No. 12/507,043 (filed on Jul. 21, 2009), and in U.S. Provisional Application Ser. Nos. 61/178,895 (filed on May 15, 2009) and 61/244,831 (filed on Sep. 22, 2009), all of which are incorporated herein by reference in their entirety. As described above, a self-sealing tissue tract does not need interventional devices or methods to help it seal—by definition, it seals by itself. For example, a self-sealing tissue tract does not need a plug, energy, sealants, clips, sutures, or the like to help it seal. It should be understood, however, that the devices, methods and kits described here may be complemented by the use of one or more additional closure mechanisms or techniques (e.g., closure devices, delivery of energy, application of pressure, etc.). For example, in some variations, compression may be applied to achieve hemostasis.

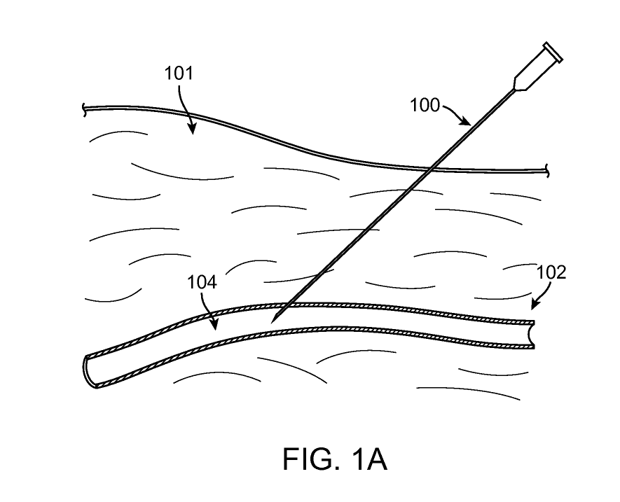

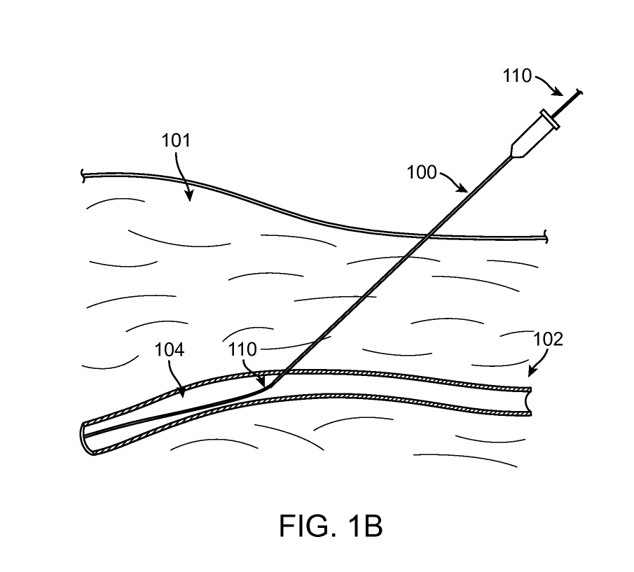

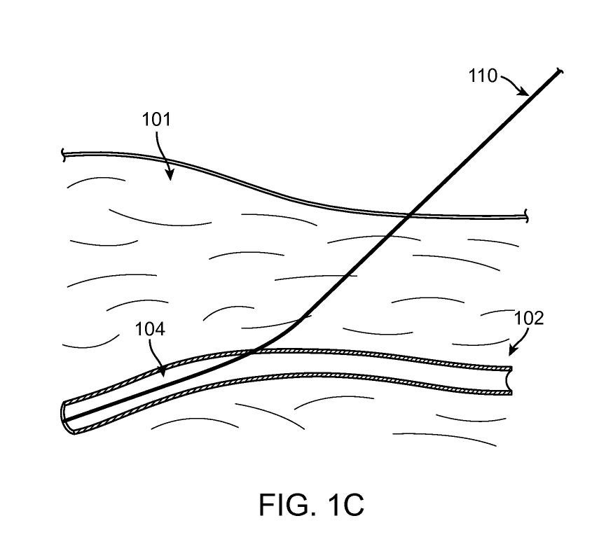

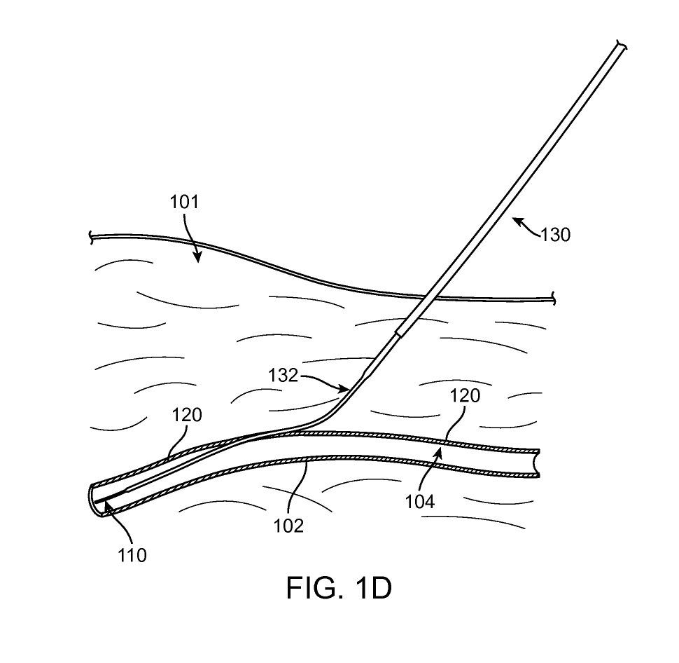

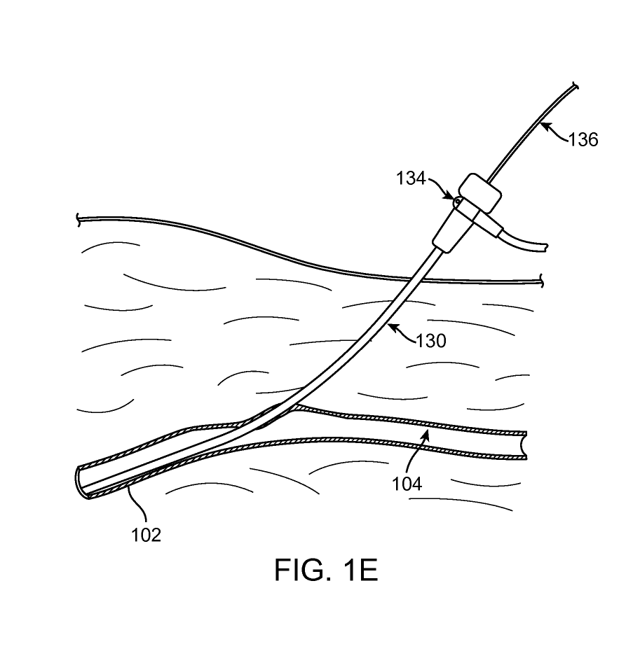

As discussed above, in some cases it may be desirable to form one or more tracts though tissue. For example, it may be desirable to form a tract through a tissue wall, such as a vessel wall, so that one or more tools may be advanced through the tract during a procedure. FIGS. 1A-1E depict one variation of a method for forming a tissue tract and advancing one or more tools through tissue.

First, FIGS. 1A-1C show a procedure for placement of a wire through a tissue. As shown in FIG. 1A, a needle (100) may be advanced through subcutaneous tissue (101) and into a lumen (104) of an artery (102). While needle (100) is depicted as having a particular configuration, it should be understood that a needle having a different configuration, or even a different type of tissue-piercing member, may be used with this method as appropriate. For example, one or more of the devices described below may be used similarly to needle (100).

Entry into lumen (104) by needle (100) may optionally be visually confirmed by observing a flash of blood (i.e., blood flow) through the needle. FIG. 1B shows advancement of a wire (110) through needle (100) and into lumen (104) of artery (102). After placement of wire (110), the needle may be withdrawn proximally, leaving wire (110) in lumen (104), as shown in FIG. 1C. Once wire (110) has been placed in lumen (104), wire (110) may be used to position one or more devices and/or tools in lumen (104). For example, FIG. 1D shows advancement of a sheath (130) over wire (110) for introduction of one or more tools therethrough.

As shown in FIG. 1D, sheath (130) is slidably coupled to a dilator (132). The dilator may be advanced through the lumen of sheath (130), and may be used to facilitate positioning of the sheath in lumen (104) of artery (102) (or other tissue as the case may be). Dilator (132) has an elongated tip, with a distal cross-sectional diameter that is smaller than the cross-sectional diameter near its proximal end. This type of sheath/dilator system may be particularly advantageous, for example, if sheath (130) has a much greater cross-sectional diameter (e.g., 5 Fr-12 Fr) than the wire (e.g., 0.012 inch to 0.35 inch) over which it will be advanced, since the wire may not provide sufficient structural support for insertion of the sheath. Here, the end of dilator (132) having a smaller cross-sectional diameter is more easily advanced over wire (126) and thus provides better support for the larger diameter portions to follow. In this way, the cross-sectional area of the tract is gradually increased, which may help in reducing the likelihood of trauma to the tissue.

FIG. 1E shows sheath (130) in lumen (104) after dilator (132) has been withdrawn. Also shown there is the proximal end (134) of the sheath having an opening therein for introduction of one or more tools (136). After the desired tool or tools have been advanced through sheath (130) and the desired procedure or procedures have been performed, the sheath and tools may be withdrawn.

In some cases, it may be desirable or advantageous to use one or more devices or methods to locate tissue prior to forming one or more tracts in the tissue. This may, for example, allow for enhanced accuracy, predictability and/or reproducibility in tract formation, and may also result in an improved overall outcome for a procedure. In certain variations, a tissue-locating device may also be capable of forming one or more tracts in tissue. Thus, the same device may be used both to locate target tissue, and to form a tract in the target tissue once it has been located. This may, for example, result in reduced procedure time, and/or a lower likelihood of complications.

In some variations, a device may comprise one or more optical components that may be used to locate tissue. Non-limiting examples of such optical components include scopes (e.g., rigid scopes or flexible scopes, such as fiber scopes), cameras, optical lenses, and the like. In certain variations, a device may comprise multiple optical components. At least some (e.g., all) of the optical components may be the same as each other, or all of the optical components may be different from each other. For example, a tissue-locating device may comprise multiple scopes (e.g., in an array), where the scopes are all identical.

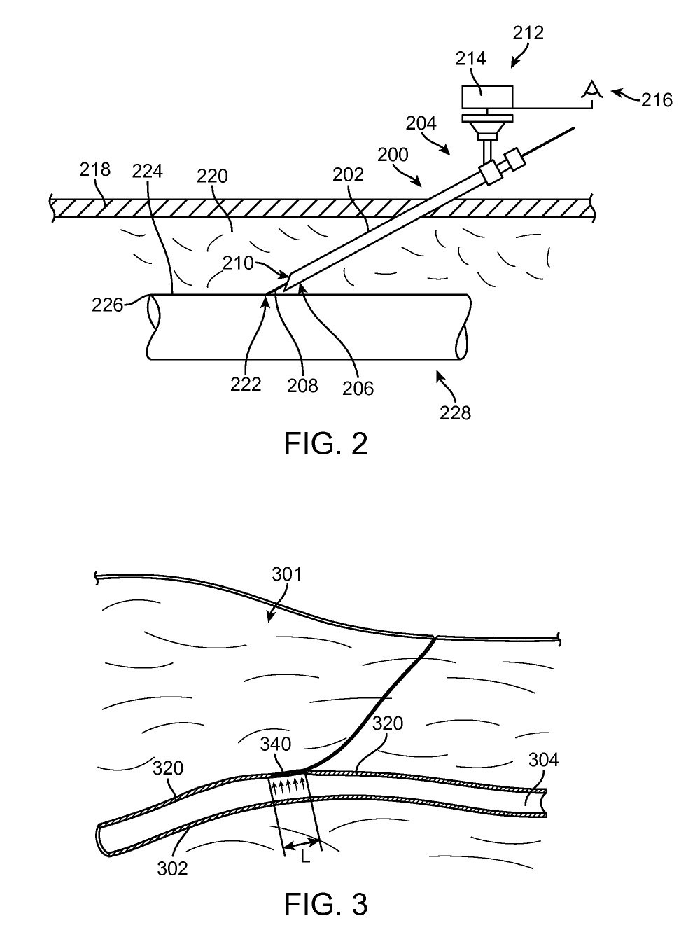

FIG. 2 depicts an exemplary variation of a device comprising a scope that may be used to locate tissue. As shown there, a tissue-locating device (200) comprises a small elongated scope (202) having a proximal portion (204) and a distal portion (206). Scope (202) may be, for example, any rigid or non-rigid scope (e.g., a flexible scope, such as a flexible fiber scope) with a lens having a range of 0° to 180° (e.g., 0° to 170° and/or a diameter of up to 10 millimeters (e.g., 1 millimeter or less). Exemplary providers of such scopes include Storz, Stryker, Olympus, Circon, R. Wolf, etc. In some variations, a scope or other optical component may be at least partially contained within a protective housing, and/or may be at least partially coated with a protective coating material.

Device (200) also comprises a tissue-piercing member (208) that is retractable and extendable from a port (210) in scope (202). It should be understood that any of the devices described here may comprise one or more tissue-piercing members, as appropriate. Further deployment of tissue-piercing member (208) through port (210) may be effected using, for example, one or more pull wires or other controls, or may even be effected by the operator manually advancing tissue-piercing member (208) distally. Other suitable deployment mechanisms may also be used. Additionally, in some variations, tissue-piercing member (208) may also be retractable relative to scope (202).

The size of scope (202) may be selected, for example, so that the scope may form a suitable initial stick through a skin surface without resulting in significant leakage, and/or so that the scope may complete multiple sticks without having a substantial adverse effect on the subject. In some variations, scope (202), or any of the other device components described herein that are configured to pierce through a skin surface without necessarily piercing through a vessel wall, may have a cross-sectional dimension (e.g., a diameter, such as an outer diameter) of, for example, up to 10 millimeters (e.g., 1 millimeter to 10 millimeters, 3 millimeters to 8 millimeters, 4 millimeters to 6 millimeters, or 1 millimeter or less). In certain variations, scope (202) may have a length of about 0.01 inch to about 6 inches (e.g., about 0.05 inch to about 3 inches). The length of a scope of a tissue-locating device may depend, for example, on the characteristics of the anatomy in which the device is to be used. Other factors may alternatively or additionally apply.

As shown, device (200) also includes an eyepiece (212) coupled to proximal portion (204) of scope (202), where the eyepiece is connected to a camera (214). An eye (216) is shown for illustrative purposes (i.e., to depict how the operator could view tissue through the scope). Examples of cameras which may be appropriate include scope cameras provided by Stryker, Pentax, Storz, R. Wolf, and the like, as well as any other suitable consumer, commercial, industrial and/or medical cameras.

Tissue-piercing member (208) may be a needle, or may be any other appropriate tissue-piercing member (e.g., a wire, energy delivery device, etc.). In cases in which tissue-piercing member (208) is a needle, the needle may be solid or hollow, may have two or more concentric needle members, may be beveled or non-beveled, and may be pointed, sharpened, or blunt. When needles are used, the needle tip may have any suitable geometry (e.g., conical, offset conical, rounded, or the like). The tissue-piercing member may be individually, discretely, or separately articulated by one or more pull wires (e.g., as described briefly above). Other appropriate actuation mechanisms may also be used. Additionally, when a tissue-piercing member is housed within a scope or another elongated member or housing, the tissue-piercing member may be sterilized and kept sterilized prior to use.

Tissue-piercing member (208), which is configured to pierce through a vessel wall, may have a cross-sectional dimension (e.g., a diameter) of, for example, up to 0.085 inch (2.16 millimeters), such as up to 0.05 inch (1.27 millimeters), or up to 0.032 inch (0.81 millimeter). Other tissue-piercing members may have different dimensions. For example, a tissue-piercing member may be substantially smaller or substantially larger in diameter, or may have any other appropriate dimensions. The dimensions of a tissue-piercing member may be selected, for example, based on the features of the target tissue.

In FIG. 2, device (200) is depicted in use. More specifically, scope (202) has been advanced through a skin surface (218) of a subject, as well as through subcutaneous tissue (220) beneath the skin surface. Tissue-piercing member (208) is extended from scope (202) and its distal end (222) is in contact with an outer surface (224) of a wall (226) of a vessel (228). Camera (214) may provide an operator looking through eyepiece (212) with an image of the scope's proximity to vessel wall (226). Assuming that vessel wall (226) is the target site, once scope (202) and tissue-piercing member (208) are in a suitable position, the desired procedure may be performed. For example, tissue-piercing member (208) may be manipulated and/or further advanced from port (210) and into vessel wall (226), to thereby form one or more tracts through vessel wall (226). The tract(s) may allow access into vessel (224) (e.g., for advancement of one or more tools during a procedure).

While scope (202) has been described as being advanced through skin surface (218), through subcutaneous tissue (220), and to vessel wall (226), in some variations, scope (202) or other device components or devices described here may be advanced through or positioned near tissue after surrounding tissue has been at least partially (e.g., fully) cut-down.

In some variations, a scope or other visualization component (or one of the other tissue-locating components described here) may be used to determine or estimate the angle of approach of a tissue-piercing member relative to a tissue wall. The operator may then perform the procedure if the angle is as desired, or may make the necessary adjustments if it is not. By tailoring the angle of approach in this way, one or more tracts having a desired configuration may be formed through the tissue. In certain variations, orientation or alignment (e.g., relative to a longitudinal axis of a vessel wall) may be determined based on identifying tissue type (e.g., fat, connective tissue, vessel wall tissue), anatomical landmarks, tissue striations, fiber directions, etc.

In some variations, as a tissue-piercing member is being advanced into and/or through tissue, the angle between the tissue-piercing member and a surface of the tissue may be from about 0° to about 180° (e.g., from about 0° to about 90°, from about 0° to about 60°, from about 0° to about 30°, from about 0° to about 20°, from about 0° to about 10°, from about 0° to about 5°, from about 1° to about 30°, from about 1° to about 20°, from about 1° to about 10°, from about 3° to about 30°, from about 3° to about 2020 , from about 320 to about 10°, from about 5° to about 30°, from about 5° to about 20°, from about 5° to about 10°, about 5°, or the like). In certain variations in which a tract is being formed in a vessel wall, the tissue-piercing member may form the above-described angle with a longitudinal axis of the vessel wall.

Some variations of tissue-locating devices may comprise one or more components that may help to provide additional information regarding orientation, location, etc. For example, a device may comprise a component that is similar to a protractor or scale, or an angle guide. The component or components may help the operator to identify the relative angle of the tissue-locating device (or one or more of its components) with respect to the body itself. In some cases, the operator may use the readings from the component(s) to adjust the device and/or tissue accordingly. The readings may, for example, be visible from the field of the scope, as determined by a feature that extends from the scope to the tissue surface. For example, in certain variations in which a tissue-locating device comprises a scope, the operator may deflect the tissue to cause the tissue to achieve a specific desired angle relative to the scope.

Tissue-locating devices may alternatively or additionally use one or more other optical components or methods to locate tissue. For example, in some variations, electromagnetic radiation of multiple wavelengths (e.g., visible light, infrared radiation (IR), etc.) may be applied to tissue, and one or more sensors may be used to measure different resulting light “signatures” and/or absorption/reflection profiles. An operator may use the measurements to identify and isolate target tissue (e.g., a vessel such as an artery) from other tissue (e.g., surrounding vessel sheath, fat, and other connective tissue). Without wishing to be bound by theory, it is believed that various tissues may have different light “signatures” and/or absorption/reflection profiles, which may allow for target tissue to be identified in this way. A device or system that is capable of providing information or measurements in this way may, for example, provide for enhanced tissue differentiation as compared to a purely visual optical device.

As discussed above, in certain variations, a self-sealing tissue tract may be formed. A self-sealing tissue tract does not need interventional devices or methods to help it seal—by definition, it seals by itself. For example, a self-sealing tissue tract does not need a plug, energy, sealants, clips, sutures, or the like to help it seal. In some cases, the angle between the tissue-piercing member and the surface of the tissue or the longitudinal axis of the tissue wall (e.g., vessel wall) may be selected to form a self-sealing tract. For example, the angle may be relatively shallow, such as less than or equal to about 30° (e.g., less than or equal to about 19°, less than or equal to about 15°, less than or equal to about 10°, less than or equal to about 5°, from about 1° to about 30°, from about 1° to about 19°, from about 1° to about 15°, from about 1° to about 10°, from about 1° to about 5°, from about 5° to about 15°, or from about 5° to about 10°).

FIG. 3 shows an exemplary self-sealing tract (340) through a vessel wall. As shown there, tract (340), which has been formed through subcutaneous tissue (301) and through a wall portion (320) of a vessel (302) having a lumen (304), is generally diagonal, and has a length (L). The length of the tract may be any suitable or desirable length to help facilitate relatively rapid sealing of the tract. For example, when the devices and methods described here are used with the vasculature, a longer tract may be desirable. This is because it is believed that, as discussed briefly above, a longer tract may expose helpful biological factors (e.g., growth factors, etc.) that may aid in sealing the tract. This may also be the case with other tissue as well. In addition, a longer tract may have a relatively large area for mechanical pressure to act on, which may cause the tract to seal more quickly. In some variations, length (L) may be greater than the thickness of wall portion (320) (e.g., in the location of wall portion (320) where tract (340) is formed). The arrows shown in FIG. 3 illustrate how pressure acting on the tract may cause the tract to seal relatively rapidly, without the need for an additional closure device. For example, the tract may seal in 15 minutes or less, 12 minutes or less, 10 minutes or less, 9 minutes or less, 6 minutes or less, 3 minutes or less, 1 minute or less, etc., reducing the duration of external compression, if any, that may be needed. Of course, if desirable, one or more additional closure devices (e.g., plugs, clips, glue, sutures, etc.) may be used.

Optical tissue-locating devices have been described above. However, some variations of tissue-locating devices may alternatively or additionally employ one or more other components or methods for locating tissue. For example, certain variations of tissue-locating devices may locate tissue via one or more ultrasonic and/or audio components, such as microphones or other acoustical sensors, or the like.

Audio components may, for example, be capable of detecting, monitoring, measuring, eco-locating, and/or triangulating the position of target tissue (e.g., a vessel wall) based on the sound of blood flow, pulsatility, etc. In some variations, a tissue-locating device may comprise multiple (i.e., at least two) audio sensors. The audio sensors may, for example, be located at the distal tip of the tissue-locating device, where they may be used to provide stereophonic direction finding at the tip. Other appropriate locations may also be used. As an example, in certain variations, two microphones may be used to triangulate and determine the point of origination of a sound. In some cases, relative intensity between sensors may be used (possibly incorporating noise-cancelling circuitry) to determine the angle or orientation of one or more device components relative to tissue.

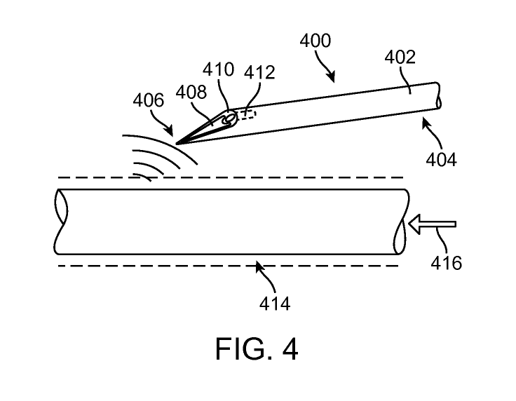

FIG. 4 shows an exemplary tissue-locating device that uses audio to locate tissue. As shown there, a tissue-locating device (400) comprises an elongated tubular member (402) (e.g., a hollow needle) having a proximal portion (404), a pointed distal end (406), and a lumen (408) therethrough. While distal end (406) is pointed (e.g., to provide enhanced tissue penetration), some variations of tissue-locating devices may comprise a distal end that is not pointed. Device (400) further comprises a wall portion (410) and a microphone (412) embedded therein, where the microphone may function essentially as a pressure sensor. More specifically, during use, microphone (412) may directly record the pressure pattern of the surrounding tissue, to thereby provide information about the surrounding tissue to the operator.

For example, and as shown in FIG. 4, as distal end (406) of elongated tubular member (402) approaches a vessel (414), microphone (412) may record a different pressure pattern resulting from the flow of blood through vessel (414) (in the direction of arrow (416)). This different pressure pattern may signal to the operator that device (400) is approaching a vessel. For example, as distal end (406) approaches vessel (414), device (400) may sense a local pressure increase resulting from pulsatility of the vessel. Alternatively or additionally, microphone (412) may allow for the operator to hear blood flowing through vessel (414) as distal end (406) approaches vessel (414). In some cases, device (400) may be especially well-applied to locating a vessel that produces a relatively high frequency measurement. In certain variations, noise cancellation methods may be applied to enhance the clarity of the signal or recording. Device (400) may be advanced through tissue to reach a target site, or in some cases may be used externally (e.g., placed on a skin surface and used to locate target tissue underneath the skin surface). Other devices described herein may also be used internally and/or externally, as appropriate.

While microphone (412) is embedded within wall portion (410) of device (400), microphones and/or other audio sensors may be incorporated into a tissue-locating device in any suitable manner. For example, in some variations, an audio sensor may be mounted to an elongated member of a tissue-locating device (e.g., via adhesion, welding, etc.). Moreover, an audio sensor may be positioned in any appropriate location of a tissue-locating device. The location of an audio sensor may depend, for example, on the anticipated location of the target tissue.

Elongated tubular member (402) may comprise any appropriate material or materials. For example, elongated tubular member (402) may comprise one or more stainless steels (e.g., 304, 304L, 316, 316L, 440C, or the like), titanium alloys (e.g., 6A1-4V or the like), nickel-titanium alloys (e.g., Nitinol), cobalt-chromium alloys (e.g., ELGILOY® alloy (Elgiloy Specialty Metals, Elgin, Ill.), MP35N® nickel-cobalt-chromium-molybdenum alloy (SPS Technologies, Inc, Jenkintown, Pa.), PHYNOX® cobalt-chromium-nickel alloy (Imphy Ugine Precision, France), or the like), metals (e.g., aluminum), and/or polymers (e.g., acrylonitrile-butadiene-styrene (ABS), nylon, acetal, high-density polyethylene (HDPE), low-density polyethylene (LDPE), polyester, polyurethane, polypropylene, other polyolefins, urethane, silicone, polyvinylchloride (PVC), polycarbonate, polyetherimide (PEI), polyethersulfone, polyarylethersulfone, polysulfone, ultra high molecular weight polyethylene (UHMWPE), polyetheretherketone (PEEK), polyetherketoneketone (PEKK), PEBAX® polyether block amide (Colombes Cedex, France), polytetrafluoroethylene (PTFE), or any other polymer, polymer blend, or filled polymer). Filled polymers may comprise, for example, glass fibers, carbon fibers, and/or other suitable carbon-based materials. Additionally, some polymers may comprise any compound/agent appropriate for improving the polymer's radiopacity, such as barium sulfate, platinum, gold, tungsten, or the like. While these materials have been described with reference to elongated tubular member (402), they may also be used in other elongated members or device components described herein, as appropriate.

In certain variations, elongated tubular member (402) may also be made to have one or more scalloped or contoured edges (e.g., top, bottom, side) to help impart flexibility. It should also be understood that elongated members described herein may have cross-sections having any suitable geometry, including but not limited to circular cross-sections.

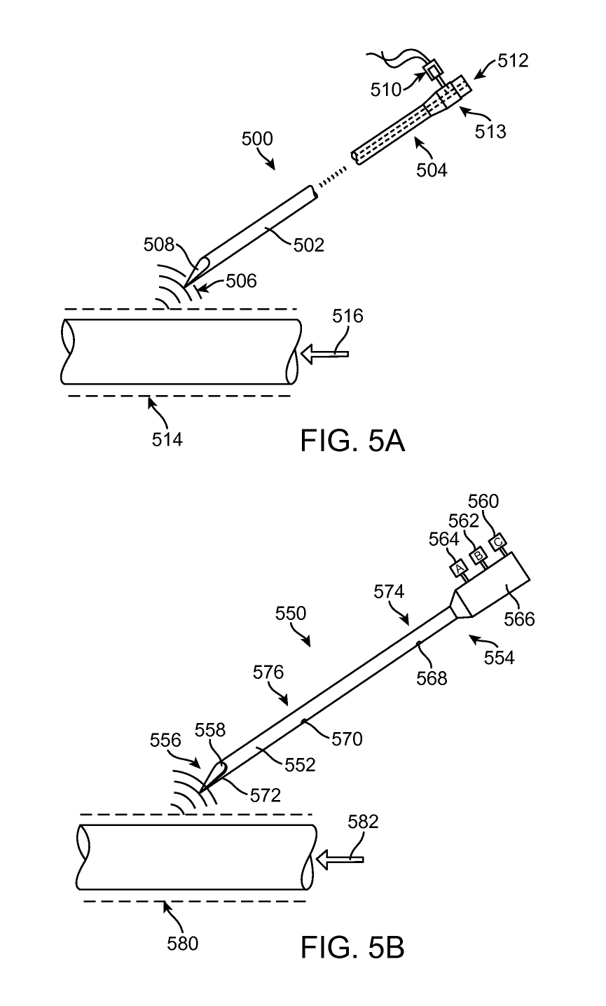

In some variations, an audio sensor may be positioned in a proximal portion of a device. As an example, FIG. 5A shows a tissue-locating device (500) comprising an elongated tubular member (502) (e.g., a hollow needle) having a proximal end (504), a pointed distal end (506), and a lumen (508) therethrough. Device (500) further comprises a microphone (510) coupled to proximal end (504) of elongated tubular member (502), as well as a valve (512) coupled to microphone (510). Valve (512) may serve as a hemostatic valve that may prevent or minimize blood flow, while also allowing guidewires, cannulas, and/or other instruments to be placed within lumen (508) and to advance devices down elongated tubular member (502). In certain variations, valve (512) and/or microphone (510) may have a feature or stopcock (not shown) that allows for air to be bled from lumen (508). While a valve is shown, certain variations of tissue-locating devices may alternatively or additionally comprise a distal portion comprising a seal and/or stopcock, or a distal portion having any other appropriate configuration.

The location of microphone (510) in a proximal portion (513) of device (500) may be especially appropriate, for example, in cases in which the microphone is not to be advanced into a subject's body during use—for example, when an expensive and/or highly sensitive microphone is used, and it is desirable to protect the microphone from body tissues or fluids. It may also be beneficial when the microphone or other audio sensor is relatively large or bulky. Similarly to microphone (412) described above, microphone (510) may record the pressure pattern of surrounding tissue as device (500) is advanced within a body of a subject. More specifically, microphone (510) may record the pressure pattern as it is transmitted up lumen (508) of elongated tubular member (502). As shown in FIG. 5A, as distal end (506) of elongated tubular member (502) approaches a vessel (514), microphone (510) may function as a pressure sensor, providing the operator with data reflecting the change in pressure as a result of blood flowing through vessel (514) in the direction of arrow (516).

In some variations, a device may comprise multiple sensors. The sensors may all be of the same type (e.g., all audio, all visual, etc.), or at least some of the sensors may be different from each other. For example, FIG. 5B shows a tissue-locating device (550) comprising an elongated tubular member (552) (e.g., a hollow needle) having a proximal end (554), a pointed distal end (556), and a lumen (558) therethrough. Device (550) also comprises multiple audio sensors (560), (562) and (564), such as microphones, coupled to or positioned within a housing (566) that, in turn, is coupled to proximal end (554) of elongated tubular member (552). Audio sensors (560), (562) and (564) are interfaced with ports (568), (570) and (572), respectively, that are located along the length of elongated tubular member (552). More specifically, port (568) is located in a proximal portion (574) of elongated tubular member (552), port (570) is located in a mid-shaft portion (576) of elongated tubular member (552), and port (572) is located at the distal end (556) of elongated tubular member (552). In certain variations, one or more microchannels, tracts, lumens and/or other conduits may be used to interface audio sensors (560), (562) and (564) with ports (568), (570) and (572). Such microchannels, tracts, lumens, conduits and the like may be formed using, for example, electrical discharge machining (EDM), laser, etching, and/or any other suitable machining, molding or forming processes.

Of course, while device (550) includes three sensors that are interfaced with three ports, a tissue-locating device may include any appropriate number of sensors interfaced with any appropriate number of ports. Moreover, the sensors and ports may be positioned in any suitable location, at any suitable distance with respect to each other, and the sensors and ports may communicate in any configuration or order/sequence. In cases in which more than two sensors are used, the sensors and/or ports may or may not be evenly spaced from each other, and may be in any appropriate configuration, such as an array, or may even be positioned irregularly with respect to each other.

During use, sensors (560), (562) and (564) may function as pressure sensors, where they directly record the pressure pattern of the surrounding tissue, and thereby provide information about the surrounding tissue to the operator. For example, and as shown in FIG. 5B, as distal end (556) of elongated tubular member (552) approaches a vessel (580), sensors (560), (562) and (564) may record a different pressure pattern resulting from the flow of blood through vessel (580) (in the direction of arrow (582)). This different pressure pattern may signal to the operator that device (550) is approaching a vessel. The presence of multiple sensors in device (500) may provide a way to differentiate relative intensities. For example, as sensor (564) increases in its vibration or pressure measurement, it may provide the operator with an indication that elongated tubular member (552) is getting close to a vessel. Additionally, the operator may notice a difference as compared to the readings or measurements from sensors (560) and (562).

In some variations, a tissue-locating device may employ ultrasonography, such as Doppler ultrasonography, to determine whether it is approaching target tissue. As an example, ultrasound imaging of a vessel wall and a device component (e.g., a tissue-piercing member) may be used to provide a live display of the position of the device component relative to the vessel wall. Ultrasound may be done internally and/or externally. In some variations, Doppler ultrasonography may be used to access whether blood is flowing through a vessel in a direction toward or away from a device. Doppler ultrasonography may also provide additional information about the surrounding tissue to the operator.

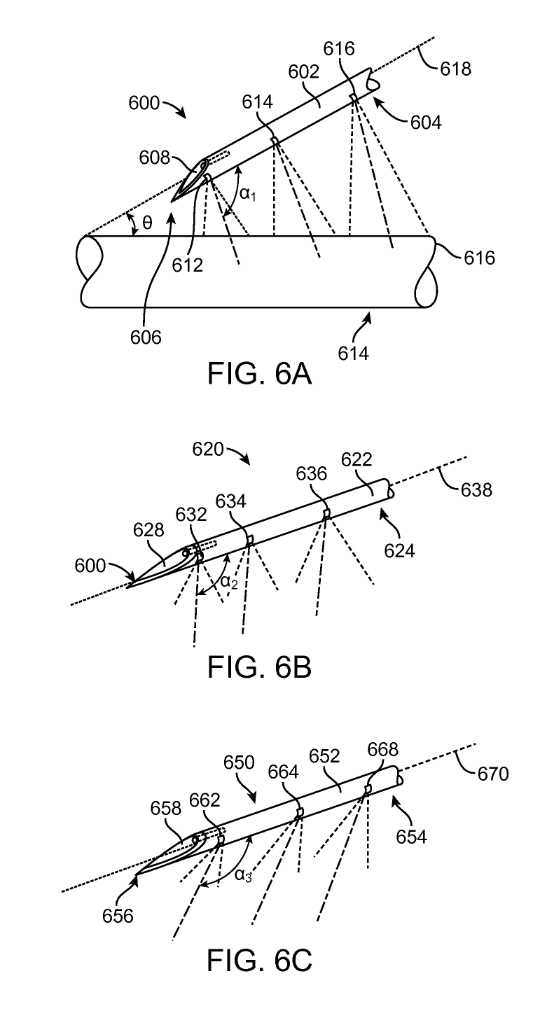

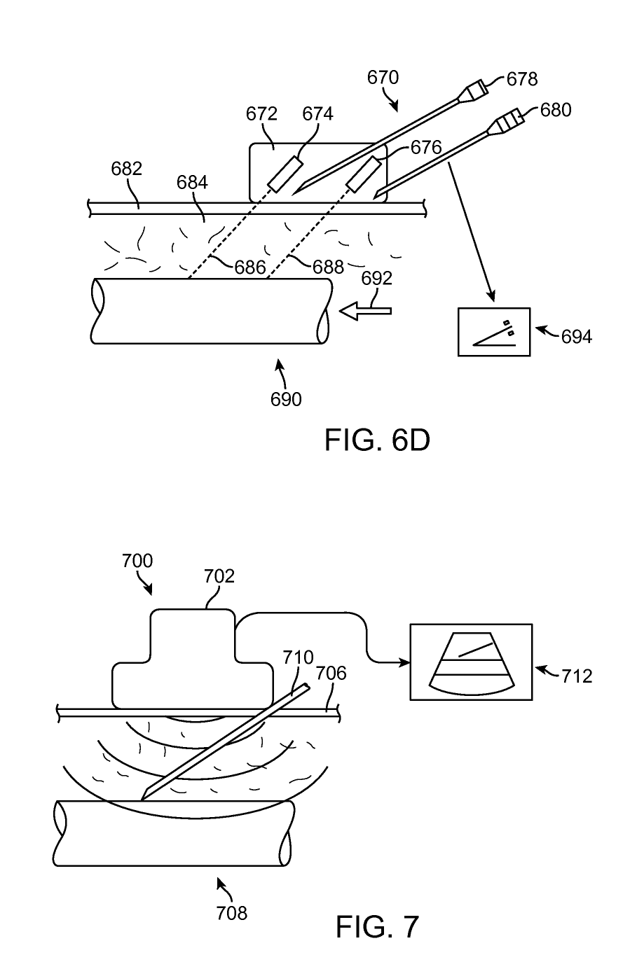

As an example, FIG. 6A shows a tissue-locating device (600) comprising an elongated tubular member (602) (e.g., a hollow needle) having a proximal portion (604), a pointed distal end (606), and a lumen (608) therethrough. Device (600) also comprises an outer array of ultrasound sensors (612), (614) and (616) along the length of elongated tubular member (602), where the outer array is normal to the longitudinal axis (618) of elongated tubular member (602). In other words, angle (α₁) (between sensor (612) and longitudinal axis (618)), and the corresponding angles for the other ultrasound sensors, are all 90°. As shown in the figure, the array of sensors may be used to notify the operator that device (600) is approaching target tissue—here, a vessel (614)—during use. FIG. 6A depicts elongated tubular member (602) as approaching a wall portion (616) of vessel (614) at an angle (θ). In some variations, angle (θ) may be less than or equal to about 30° (e.g., less than or equal to about 19°, less than or equal to about 15°, less than or equal to about 10°, less than or equal to about 5°, from about 1° to about 30°, from about 1° to about 19°, from about 1° to about 15°, from about 1° to about 10°, from about 120 to about 5°, from about 5° to about 15°, or from about 5° to about) 10°.

In use, device (600), and other devices described herein, may be advanced through tissue directly, or may be advanced through tissue by being advanced within or over one or more other devices, such as a catheter. In some variations, device (600) may be in the form of a component located at a distal end of another device. For example, device (600) may be in the form of a component that is coupled to a distal end of a catheter. In certain variations, a device described herein may be deployable from another device. For example, a device may be slidably housed within, and deployable from, an elongated member.

While an ultrasound sensor array that is normal to the longitudinal axis of an elongated member of a tissue-locating device has been described, some variations of devices may comprise ultrasound arrays that are positioned differently. For example, in certain variations, a tissue-locating device may comprise one or more ultrasound sensors that are configured to point in the direction of blood flow through a vessel during use. This positioning may, for example, enhance the function of the sensors. As an example, FIG. 6B depicts a tissue-locating device (620) comprising an elongated tubular member (622) (e.g., a hollow needle) having a proximal portion (624), a pointed distal end (626), and a lumen (628) therethrough. Device (620) also comprises an outer array of ultrasound sensors (632), (634) and (636) along the length of elongated tubular member (622), where the outer array is angled toward distal end (626) of elongated tubular member (622). More specifically, each of ultrasound sensors (632), (634) and (636) is positioned at an angle (α₂) relative to the longitudinal axis (638) of elongated tubular member (622), where angle (α₂)>90°. For example, in certain variations, angle (α₂) may be greater than about 90° (e.g., at least about 100°, at least about 110°, at least about) 120° and/or less than or equal to about 135° (e.g., at most about 120°, at most about 110°, at most about 100°). While not shown, in some variations, device (620) may further comprise an additional ultrasound sensor located at its distal end (626). Additionally, in certain variations, a device may comprise multiple ultrasound sensors, where at least one of the sensors is angled differently from at least one of the other sensors.

Ultrasound arrays may be even more angled with respect to other components of a tissue-locating device. As an example, FIG. 6C shows a tissue-locating device (650) comprising an elongated tubular member (652) (e.g., a hollow needle) having a proximal portion (654), a pointed distal end (656), and a lumen (658) therethrough. As with device (620) above, device (650) also comprises an outer array of ultrasound sensors (662), (664) and (668) along the length of elongated tubular member (652), where the outer array is angled toward distal end (656) of elongated tubular member (652). However, the outer array is positioned at an angle (α₃) relative to the longitudinal axis (670) of elongated tubular member (652), where angle (α₃)>>90°. For example, in some variations, angle (α₃) may be from about 135° to about 180° (e.g., from about 150° to about) 165°.

The arrangement of ultrasound sensors or other appropriate sensors with respect to one or more of the other components of a tissue-locating device may depend, for example, on the configuration, size and/or shape of the other component(s), and/or on the type of tissue being targeted. Other factors may also apply. Moreover, the arrangement or positioning of ultrasound sensors with respect to each other may vary depending, for example, on the required function of the device. In certain variations, a single ultrasound sensor or a plurality of ultrasound sensors may be directly affixed to the distal tip of a tissue-locating device or device component, and/or along the length of the device or component, and may be used to provide distance information and possibly orientation information relative to the target tissue (e.g., a tissue wall). It should be noted that ultrasound sensors do not necessarily need to all be aligned along a device in order to provide location information. Additionally, in some variations, ultrasound sensors may be positioned so that they may be used to determine device orientation with respect to tissue. Ultrasound sensors may also sense relative intensities based on their location. In some cases, the position of a tissue-locating device may be adjusted based on input from the device's ultrasound sensors. For example, if a particular sensor is sensing better than other sensors, then the position of the device may be adjusted (e.g., to better align the particular sensor with the other sensors).

Tissue-locating devices may enter the body during use, or in some cases, may remain partially or entirely external to the body during use. As an example, FIG. 6D depicts a tissue-locating device (670) comprising a probe housing (672) having two ultrasound probes (674) and (676), as well as two needle probes (678) and (680). Ultrasound probes (674) and (676) are fixed in position relative to probe housing (672) and provide a vessel angle, through a software measurement/calculation, relative to the skin surface when device (670) is in use. Needle probes (678) and (680) may be oriented through an angle measurement feature (not shown) that senses the angle of needle probes (678) and (680) relative to probe housing (672). During use, and as shown, housing (672) may be positioned adjacent a skin surface (682) of a subject, whereby ultrasound probes (674) and (676) are used to calculate the vessel angle, such that needle probes (678) and/or (680) may be advanced through skin surface (682), and through subcutaneous tissue (684). Paths (686) and (688) indicate the ultrasound probes' line-of-sight relative to the tissue. Ultrasound probes (674) and (676) may interface with an output display (694) to provide the operator with information about the presence of vessel (690), as well as its position (angle) with respect to the needle probes.

Other variations of devices that employ Doppler ultrasonography may be used to locate tissue. For example, FIG. 7 shows a tissue-locating device (700) comprising an ultrasound imaging probe (702). During use, and as shown, probe (702) may be positioned on a skin surface (706) and may be used to determine the location of target tissue—here, a vessel (708). Once the target tissue has been identified, a tissue-piercing member (710), such as a needle, may be advanced through skin surface (706) and to the target tissue. Thus, tissue-locating device (700) may remain entirely external to the body during use. This may be advantageous, for example, in terms of limiting the invasiveness of a procedure, as well as limiting procedure time and the likelihood of harm to the subject.

While tissue-piercing member (710) is depicted as a separate component from device (700), some variations of devices comprising ultrasound imaging probes may also comprise one or more tissue-piercing members that are a component of the device.

In certain variations, and as shown, probe (702) may be connected to a screen output, such as a monitor (712), which may display the ultrasound images to the operator. In this way, monitor (712) may be used to help position tissue-piercing member (710) prior to and/or during tract formation. While not shown in FIG. 7, in some variations, a tissue-piercing member may be advanced through a probe and into target tissue, or may be advanced through or along one or more other components or positions of a tissue-locating device. In certain variations, the position of a tissue-piercing member relative to a probe may be measured by one or more sensors. The sensor(s) may, for example, determine the angle of the tissue-piercing member relative to the probe. In some variations, this positional information may be provided as feedback into software that processes signals from the ultrasound probe.

In certain variations, a device or method may use an ultrasound probe and accompanying software (e.g., A-Trace) that provides an ultrasound trace in which tissue features, boundaries, etc. are identified by peaks and valleys. In other words, the software may be used to identify the tissue boundaries graphically, thereby providing a type of quantitative evaluation of tissue. Essentially, the device may be capable of providing a two-dimensional graphical representation of an ultrasound reading, having peaks and valleys, in which each peak may correlate with a tissue structure. In some instances, traces from multiple sensors may be compared against each other to help locate and/or identify tissue.

In certain variations, a curved tissue-piercing member, such as a curved needle, may include one or more ultrasound sensors. The curved shape of the tissue-piercing member may, for example, make it relatively easy to re-direct or otherwise manipulate the tissue-piercing member, in response to input from the ultrasound sensors. Of course, while ultrasound sensors have been described, a curved tissue-piercing member may alternatively or additionally comprise one or more other types of sensors.

In some variations, a device may employ one or more movable ultrasound transducers to locate target tissue. The movable ultrasound transducers may, for example, be capable of rotating and/or oscillating. In other words, the device may comprise one or more scanning ultrasound transducers that move to collect an image. As an example, a rotating ultrasound transducer may be incorporated in a ring around a catheter, and may be used to provide a 360° image of the environment around the catheter as the catheter is being advanced to a target site, and/or while the catheter is located at a target site. Other devices having other appropriate configurations may also be used. Additionally, it should be noted that a device may, of course, comprise one or more stationary transducers, such as a phased array (i.e., multiple stationary transducers). As an example, a device may comprise or one or more fixed ultrasound transducers incorporated in a ring around a catheter. A device may comprise one or more stationary transducers either in addition to, or as an alternative to, comprising one or more movable transducers.

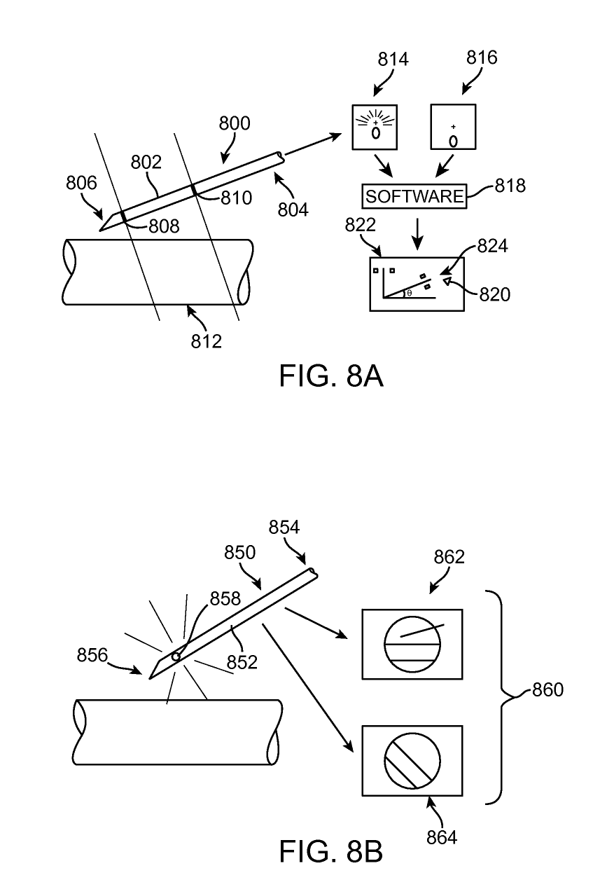

An example of a tissue-locating device comprising at least one scanning ultrasound transducer is shown in FIG. 8A. As shown there, a tissue-locating device (800) comprises an elongated tubular member (802) (e.g., a needle) having a proximal portion (804) and a pointed distal end (806), and two ultrasound transducer rings (808) and (810) located along elongated tubular member (802). During use, and as shown in FIG. 8A, as pointed distal end (806) of elongated tubular member (802) approaches a vessel (812) after piercing through a skin surface (not shown), image output (814) and (816) from ultrasound sensors (808) and (810) (respectively) may be processed by software (818), which may be in communication with an output display (820), such as a monitor. Output display (820) may display an image that, for example, shows the axial alignment (822) relative to vessel (812), as well as the angle (824) relative to the vessel axis. By utilizing the position of the vessel cross-section within each image as generated from each transducer ring, the software can calculate the angle of tissue-locating device (800) relative to vessel (812), as well as the axial alignment of tissue-locating device (800) relative to vessel (812). This information may help the operator to ultimately achieve highly accurate tissue tract formation. For example, in some variations in which device (800) further comprises a tissue-piercing member, the operator may use the image on output display (820) to align device (800) in a particular position and orientation to form the desired tissue tract.

Other variations of devices employing one or more movable ultrasound transducers may alternatively or additionally be used. As an example, FIG. 8B shows a tissue-locating device (850) comprising an elongated tubular member (852) (e.g., a needle) having a proximal portion (854) and a pointed distal end (856), as well as a planar ultrasound transducer (858), which may comprise several discrete fixed arrays of ultrasound transducers (where the ultrasound transducer imaging plane contains the longitudinal axis of elongated tubular member (852)). Device (850) is in communication with an output display (860), such as a monitor, which may provide a superimposed image (862) and/or a raw image (864) of the target site and the device to the operator. Superimposed image (862) depicts a graphical representation of elongated tubular member (852) in a fixed position relative to a moving vessel wall, while raw image (864) is the actual image as generated from planar ultrasound transducer (858).

In some variations, a tissue-locating device may use electrical impedance measurements to locate target tissue. For example, an array of sensors may be used to measure variations in impedance to determine tissue type and/or to differentiate tissue (e.g., vessel tissue vs. surrounding tissue). In some cases, a distributed sensor array may then be used to determine the relative location of the tissue types. Such a device and method may advantageously be relatively simple to implement. Without wishing to be bound by theory, it is believed that impedance measurements may be lower when such a device is positioned near a vessel, since blood vessels may generally function as good electrical conductors.

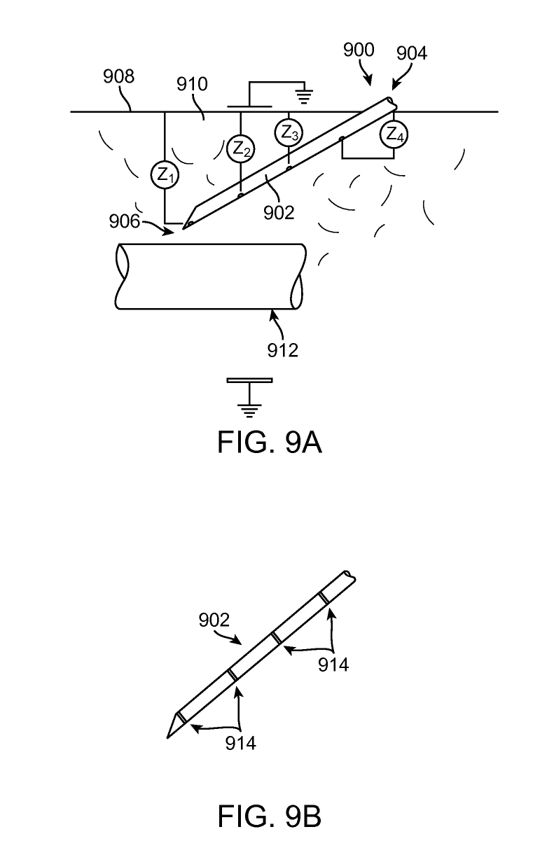

FIG. 9A depicts an exemplary device for locating tissue based on electrical impedance measurements. As shown there, a tissue-locating device (900) comprises an elongated member (902), such as a needle, having a proximal portion (904) and a pointed distal end (906). Device (900) is capable of measuring electrical impedance relative to the outer skin surface (908) through which elongated member (902) is advanced. As shown in FIG. 9A, elongated member (902) has been advanced through a skin surface (908) and through subcutaneous tissue (910). As elongated member (902) approaches a vessel (912), its electrical impedance measurements may change. This change may notify an operator that elongated member (902) is in close proximity to vessel (912). As show in FIG. 9B, in certain variations, elongated member (902) may comprise markings (914) that may provide positional information, for example, as device (900) is used to locate a target tissue.

While certain variations of devices, components and methods for locating tissue have been described, other variations may alternatively or additionally be employed.

As an example, some variations of devices may use tactile sensors to locate and/or evaluate tissue (e.g., by mechanically sensing the tissue and/or its displacement or movement). For example, devices may provide for direct measurement of vessel wall movement or displacement, or for direct measurement of tissue mechanical properties (e.g., stiffness, compliance, etc.). This may be provided, for example, by using a displacement-type sensor or mechanism, such as a linear variable differential transducer (LVDT) or a deflectable feature instrumented with a strain-gauge or force sensor.

As another example, certain variations of devices may utilize thermal sensing to locate and/or evaluate tissue. The devices may, for example, comprise one or more thermocouples, thermistors, infrared (IR) detectors, and/or other suitable components, that may provide for such thermal sensing. The thermal sensing capability may be used, for example, to detect temperature variation between a vessel wall through which blood is flowing, and the surrounding tissue. Such devices may be relatively simple, and may be relatively easy and inexpensive to manufacture and/or implement.

In some cases, devices and methods may take advantage of differential temperature measurements to identify heat transfer into the blood, and to thereby locate tissue with active blood flow (e.g., vessels or vascularized organs). In some such cases, a probe may be placed in tissue when the probe is at a higher temperature than the tissue. The probe may be heated to the higher temperature before and/or after insertion into the tissue. For example, the probe may be at a slightly elevated temperature (e.g., about 50° C.) relative to normal body temperature (i.e., 37° C.), while not being so high as to cause damage to tissue during use. The change in temperature of the probe as it is advanced through tissue may help the operator to identify areas of heat transfer in the body, and may thereby help to identify tissue in the vicinity of the probe. For example, if the probe is positioned near a vessel, blood flowing through the vessel may take some of the heat away from the probe more rapidly than non-moving tissue. The resulting drop in the probe's temperature (e.g., relative to a reference point that is slightly farther away) may then be measured.

It should be noted that in certain cases a cooled catheter or other body may be positioned in a body of a subject, and the heating of the catheter may be measured as a way to determine the location of the catheter, in a similar fashion as described with respect to the heated probe. A device may be cooled using, for example, liquid nitrogen and/or any other appropriate cooling sources. The device may be cooled to a temperature that is lower than normal body temperature (37° C.).

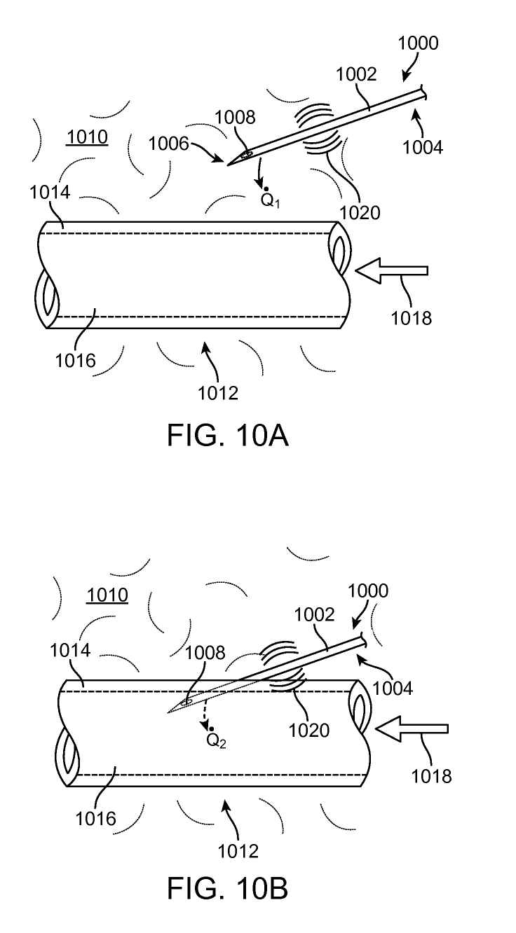

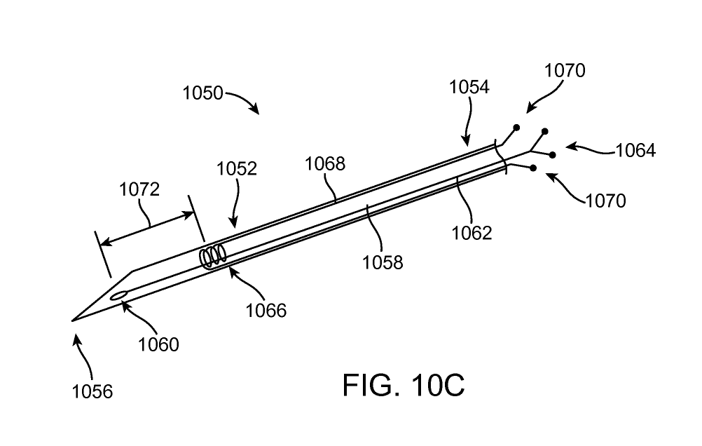

FIGS. 10A and 10B depict a tissue-locating device (1000) comprising a tissue-piercing member (1002) having a proximal portion (1004), a pointed distal end (1006), and a lumen (not shown). Device (1000) further comprises a temperature sensor (1008) located in the lumen, near distal end (1006). As shown in FIG. 10A, device (1000) is being advanced through subcutaneous tissue (1010) and toward a vessel (1012). Vessel (1012) comprises a wall portion (1014) defining a lumen (1016), through which blood flows in the direction of arrow (1018). Here, device (1000) may be heated prior to and/or during advancement through subcutaneous tissue (1010), such that heat (1020) emanates from tissue-piercing member (1002) and into the surrounding tissue. This heat flux is depicted as (Qdot₁). Referring now to FIG. 10B, as tissue-piercing member (1002) is advanced through wall portion (1014) of vessel (1012), heat continues to emanate from tissue-piercing member (1002), such that the heat flux changes and is now (Qdot₂). Thus, (Qdot₂) is greater than (Qdot₁). Temperature sensor (1008) may be used to measure the temperature of tissue-piercing member (1002) in or near distal end (1006), and may therefore provide the operator with an indication of a change in the surrounding tissue (i.e., by recording a change in this temperature). It should be noted that in cases in which tissue-piercing member (1002) is cooled prior to being advanced through tissue, the heat flux will change direction (although the magnitude of (Qdot₂) should still be greater than that of (Qdot₁)).

While one temperature sensor (1008) is shown, some variations of devices may comprise multiple temperature sensors (e.g., located at the tip of the device, near a heater element of the device, etc.). Alternatively or additionally, a device may comprise multiple heating sources and/or multiple cooling sources.

A tissue-piercing member body can contain one or more heat sources along its length that can provide fixed power outputs or have adjustable power outputs. For a device using a fixed power output, it is possible in some instances that the heat capacity of the surrounding tissue (outside or removed from the target tissue region) will be great enough that the temperature at the temperature sensor(s) is effectively body temperature. As a result, the temperature sensors may provide no real discernable differential temperature when in contact with the target tissue region or lumen (possibly containing a moving blood flow). In such cases, the fixed power output may have to be increased (depending on a specific tissue type, region, tissue composition, etc.). Alternatively or additionally, the input power may have to be adjustable (either manually or automatically), such that sufficient power may be delivered to the tissue to create a temperature gradient in the tissue, as measured by the temperature sensor(s), without being so high as to result in a temperature that can cause local damage, scarring, charring, etc. to the tissue immediately around the heat source. A temperature sensor located adjacent, within, or near to the heat source could monitor the closest tissue region to prevent an over-temperature condition that could lead to such tissue damage. The temperature sensor may be used for feedback into a control circuit in the device design that can automatically maintain a maximum safe power input.

In certain variations, a pictorial chart, heat flow plot or the like may be generated and evaluated for a determination of the location of the probe.

FIG. 10C depicts another exemplary tissue-locating device (1050) that uses thermal sensing to locate tissue. As shown there, device (1050) comprises an elongated tubular member (1052), such as a needle, having a proximal portion (1054), a pointed distal end (1056), and a lumen (1058). Elongated tubular member (1052) may be configured, for example, for passage of a guidewire through its center (e.g., when device (1050) has been located within a vessel during use). Device (1050) additionally comprises a temperature sensor (1060) (e.g., a thermocouple or thermistor, or any other appropriate temperature sensor) disposed within lumen (1058), near distal end (1056) of elongated tubular member (1052). Temperature sensor (1060) may, for example, be mounted to elongated tubular member (1052) within lumen (1058). At least two wire leads (1062) are connected to temperature sensor (1060), and extend proximally through lumen (1058), to wire terminal ends (1064). Additionally, device (1050) comprises a heater element (1066) (e.g., a heater, small resistor, wire coil, flat heating element, or the like) disposed within lumen (1058), proximal to temperature sensor (1060). While not shown here, in some variations a temperature sensor (e.g., a thermocouple, thermistor, or the like) may be located at the location of heater element (1066), and may be used to measure the temperature at that location. Heater element (1066) is connected to at least two wire leads (1068) that also extend proximally through lumen (1058), to wire terminal ends (1070). As shown, heater element (1066) and temperature sensor (1060) are separated by a distance (1072). In some variations, distance (1072) may be equal to at least about 0.2 times the diameter of elongated tubular member (1052) (e.g., from about 0.2 times the diameter of elongated tubular member (1052) to about 5 times the diameter of elongated tubular member (1052)).

During use, heater element (1066) may be used to heat the area around distal end (1056), and temperature sensor (1060) may be used to measure the temperature differential between distal end (1056) and the rest of elongated tubular member (1052). Heater element (1066) may be used to heat the area around distal end (1056) to a temperature that is greater than normal body temperature (i.e., 37° C.), such as 50° C. As distal end (1056) approaches a blood source (e.g., a vessel with blood flowing therethrough), it is believed that the amount of heat leaving distal end (1056) will increase. Temperature sensor (1060) may be used to measure the resulting decrease in the temperature of distal end (1056), and to thereby provide the operator with a signal that device (1050) is approaching a target site.

As an additional example, some variations of tissue-locating devices may employ X-ray fluoroscopy to locate tissue and/or to properly position a tissue-piercing member during tissue tract formation. For example, X-ray imaging may be used to provide a direct image of a radiopaque device (e.g., a needle comprising radiopaque markers) penetrating surrounding tissue, and then penetrating a vessel wall. In some cases, it may be difficult to determine orientation in other planes from a single planar view. In such cases, multiple views may be used, and/or other radiopaque markers may be used (e.g., to help align and/or orient a device relative to the plane of an image). In certain variations, it may be desirable to deliver contrast agent into tissue during a tissue-locating procedure. As an example, contrast agent may be delivered through a vessel to help to identify the vessel by identifying blood within a lumen of the vessel.

Of course, it should be understood that any appropriate combination of imaging and/or sensing modalities, including any of those described herein, may be employed, as well. For example, in some variations, a tissue-locating device may comprise both an optical sensor and a tactile feature, which may be used together to help locate and identify target tissue.

In some cases, a device may be used to position and/or otherwise mechanically manipulate tissue prior to and/or during the formation of one or more tracts in the tissue. This manipulation of the tissue may, for example, provide enhanced control over, and accuracy in, tissue tract formation. The device may or may not also be able to serve one or more other functions. For example, the device may also be capable of locating target tissue (e.g., using one or more of the methods described herein), and/or may be capable of forming one or more tracts in tissue.

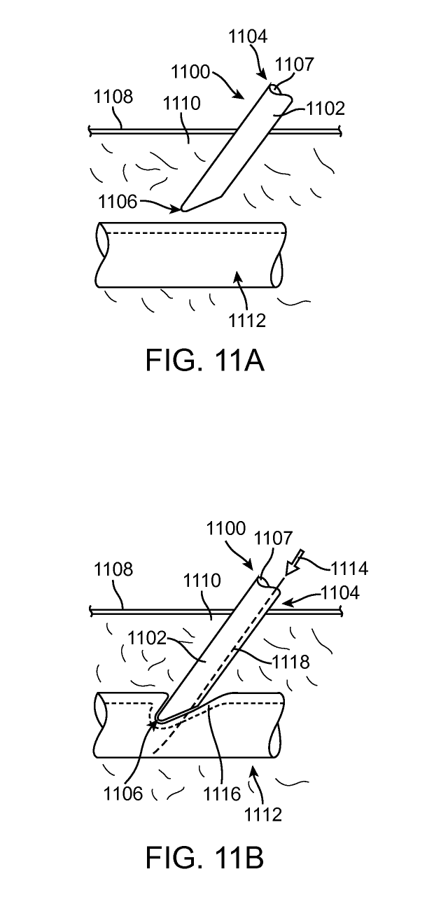

FIGS. 11A and 11B show an exemplary variation of a device (1100) that may be used to position tissue and/or to provide enhanced control over tissue for the purposes of tract formation in the tissue. As shown there, device (1100) comprises an elongated member (1102) having a proximal portion (1104), a blunt distal end (1106), and a lumen (1107) therethrough. While many of the devices described here comprise a lumen, some variations of devices may comprise multiple lumens, and certain variations of devices may not comprise any lumens at all.

Referring first to FIG. 11A, device (1100) may be advanced through a skin surface (1108) and through subcutaneous tissue (1110), until blunt distal end (1106) of elongated member (1102) reaches a vessel (1112). Referring now to FIG. 11B, a force may be exerted upon proximal portion (1104) of elongated member (1102), in the direction of arrow (1114), so that blunt distal end (1106) contacts a wall portion (1116) of the vessel and substantially deforms or deflects the wall portion. As a result, the tissue may tightly approximate the surface of distal end (1106). While the tissue is approximated in this way, a tissue-piercing member (not shown) may be advanced through lumen (1107) of elongated member (1102) (e.g., along a path (1118)). The tissue-piercing member may then be advanced through vessel wall portion (1116), thereby forming an angled path through the vessel wall portion.

By substantially deforming or deflecting vessel wall portion (1116), device (1100) may, for example, position the tissue for a tissue-piercing member to be advanced therethrough at a desired angle. In some cases, elongated member (1102) may push one side of a vessel all the way to the other side of the vessel, and may thereby temporarily block vessel flow (e.g., for a brief period of time, such as 5 seconds). This contact between opposing sides of the vessel wall may allow the surrounding tissue to substantially support the applied pressure and provide good apposition to the surface of distal end (1106). In other words, this contact may provide for good placement of the tissue against the surface of distal end (1106), and for good counter-pressure.

While device (1100) is advanced through a skin surface and into tissue beneath the skin surface, in some variations, a device may be used to deform or deflect the outer skin surface itself, without actually piercing the skin surface or otherwise being advanced through it. For example, the device may be pushed against the skin surface to deform or deflect it, thereby providing an angled approach for a tissue-piercing member to be advanced through the skin surface. In some cases, the deformation or deflection of the skin surface may result in a corresponding deformation or deflection of a target tissue wall (e.g., a target vessel wall portion) beneath the skin surface. By remaining only on the outer skin surface, the device may be relatively unlikely to cause tissue damage, and may result in a relatively short overall procedure, as well as a relatively quick recovery time.

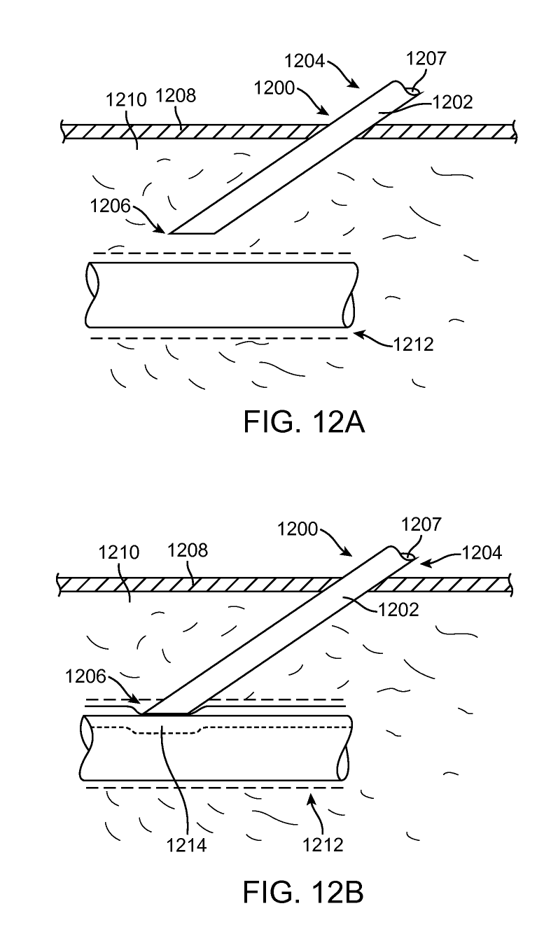

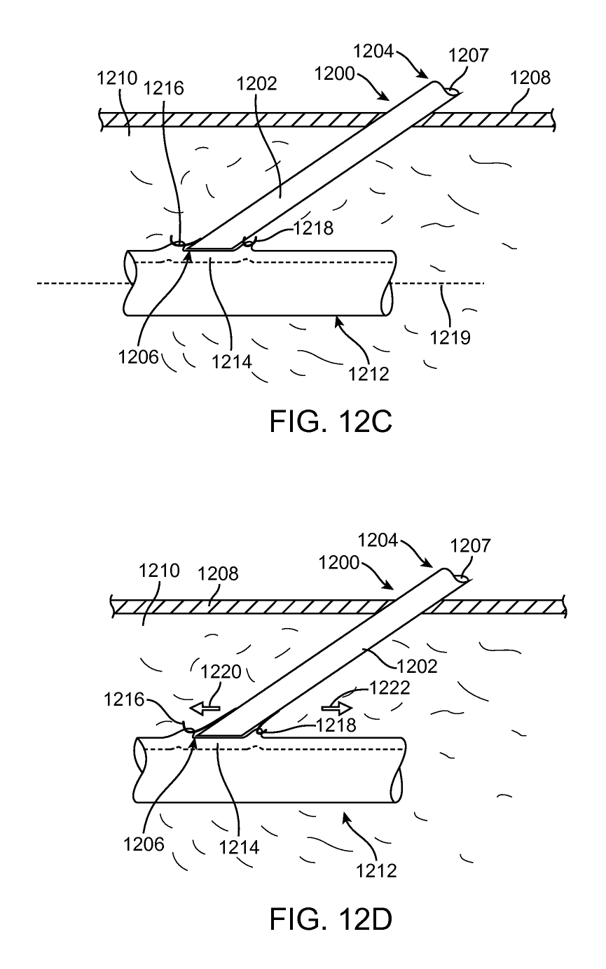

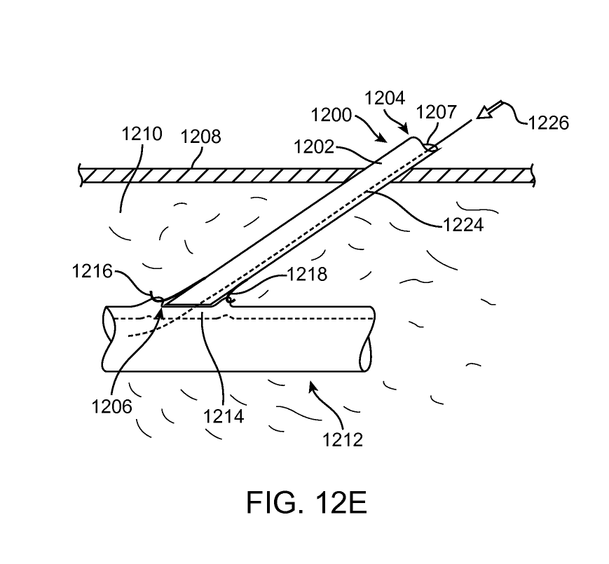

Another exemplary variation of a device that may be used to mechanically deform a vessel wall is depicted in FIGS. 12A-12E.

First, FIG. 12A shows a tissue-tensioning device (1200) comprising an elongated member (1202) having a proximal portion (1204), a pointed distal end (1206), and a lumen (1207) therethrough. As shown there, elongated member (1202) may be advanced through a skin surface (1208) and through subcutaneous tissue (1210), so that distal end (1206) is positioned near a vessel (1212).

Next, and referring to FIG. 12B, distal end (1206) of elongated member (1202) may be positioned in contact with vessel (1212), and a force may be exerted on elongated member (1202), to thereby deform a wall portion (1214) of vessel (1212). Referring now to FIG. 12C, hook members (1216) and (1218) may then be deployed from opposing sides of elongated member (1202), along a longitudinal axis (1219) of vessel (1212), so that the hook members engage and hook into wall portion (1214). It should be noted that while device (1200) includes hook members located on opposing sides of an elongated member, other variations of devices may alternatively or additionally comprise one or more other variations of tensioning members, may comprise more or fewer tensioning members, and/or may comprise tensioning members that are positioned or arranged differently from hook members (1216) and (1218). As an example, some variations of devices may comprise only one tensioning member. As another example, certain variations of devices may comprise four tensioning members. During use, each of tensioning members may tension the tissue in a direction that is approximately 90° apart from the direction of tensioning by either of its neighboring tensioning members. Enhanced radial stretching may be provided by, for example, increasing the number of tensioning members and, therefore, the number of tensioning directions.

As shown in FIG. 12D, once hook members (1216) and (1218) have engaged vessel wall portion (1214), the hook members may be actuated in different directions (i.e., in the directions of arrow (1220) and arrow (1222), respectively). This may result in a tensioning or stretching of vessel wall portion (1214), which may stabilize the vessel wall portion and effectively make it taut for highly accurate tissue penetration and tissue tract formation. As an example, FIG. 12E illustrates how a tissue tract may be formed in this case. More specifically, a tissue-piercing member (not shown) may be advanced through lumen (1207) of elongated member (1202) (e.g., along a pathway (1224), in the direction of arrow (1226)), and through vessel wall portion (1214), thereby forming a tract (e.g., a diagonal tract) through the vessel wall portion. The tract, as with other tissue tracts described herein, may be a self-sealing tract. As described above, a self-sealing tissue tract does not need interventional devices or methods to help it seal—by definition, it seals by itself. For example, a self-sealing tissue tract does not need a plug, energy, sealants, clips, sutures, or the like to help it seal. A tissue tract may be of any suitable length, and in some cases may traverse through the tissue. Once a tissue tract has been formed, one or more tools may be advanced through the tract. For example, a guidewire and/or an introducer (e.g., a 5 Fr or 6 Fr introducer) may be advanced through the tract.

While hook members (1216) and (1218) may be actuated in different directions during use, in some variations of devices and methods employing multiple tensioning members, at least one of the tensioning members may be actuated while at least one of the other tensioning members is not actuated. A tensioning member that is not actuated may, for example, be used to help stabilize the device during actuation of another tensioning member.

Any appropriate devices and methods may be used for tissue tract formation. As discussed above, in some variations a tissue-locating device may also be capable of forming one or more tracts in tissue (e.g., once the device has located target tissue). Other variations of tissue tract-forming devices may also be used.

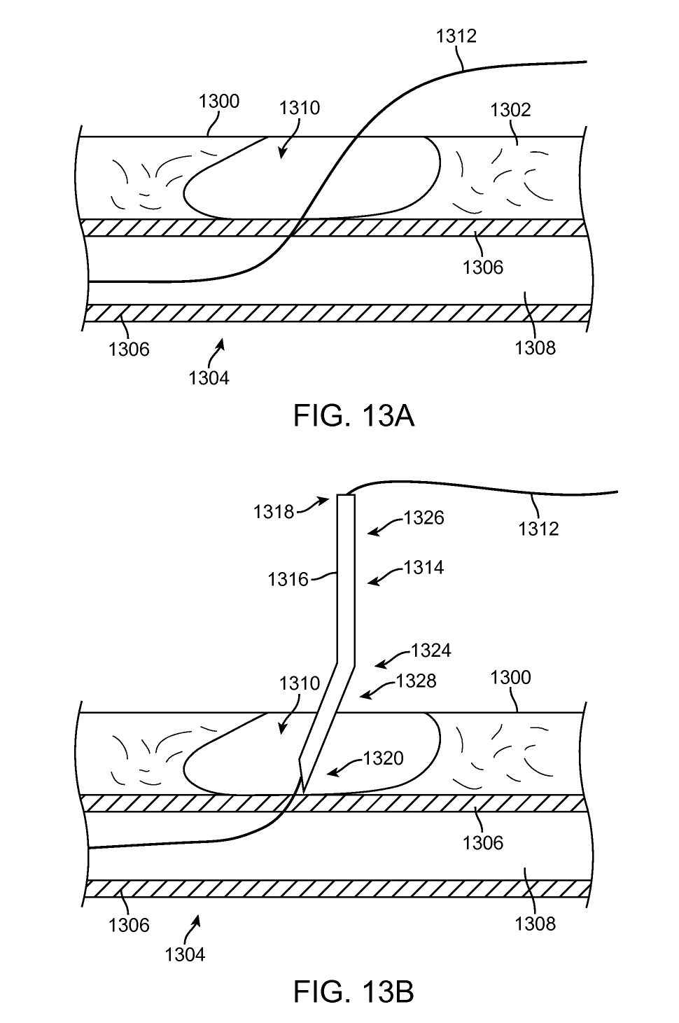

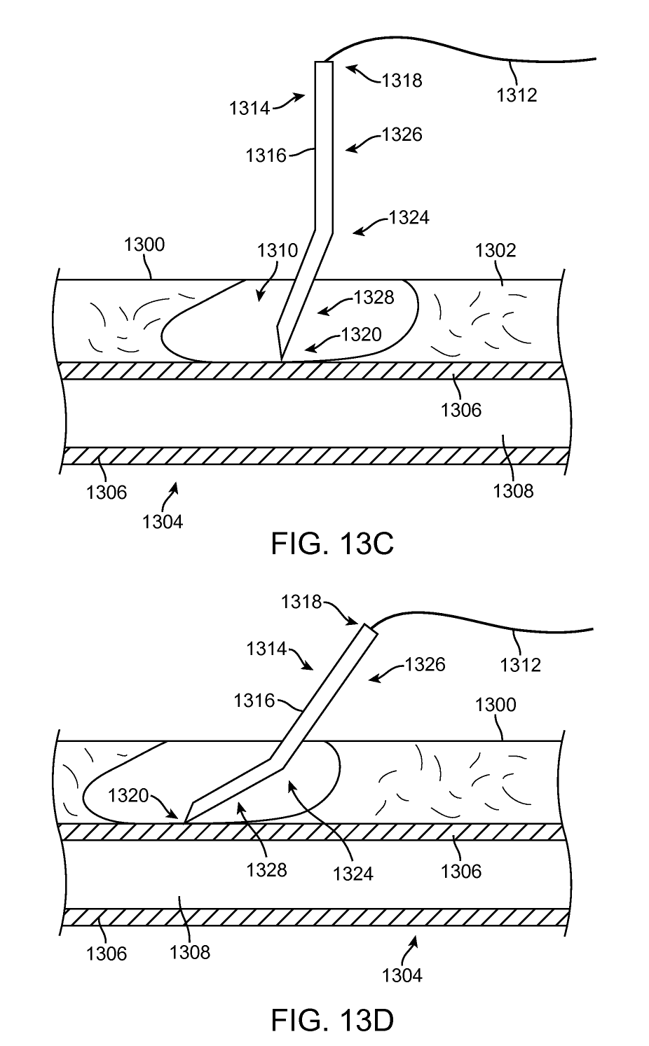

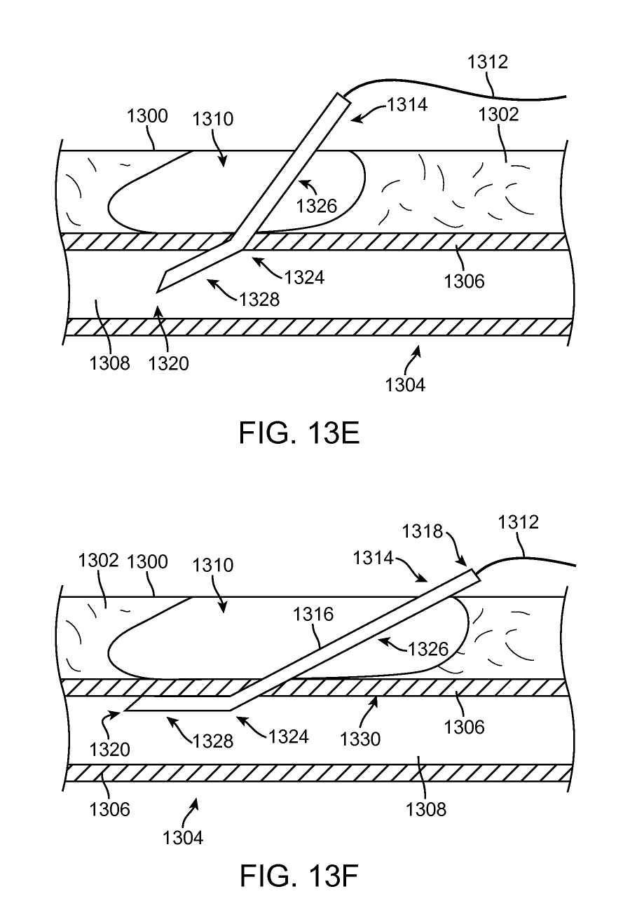

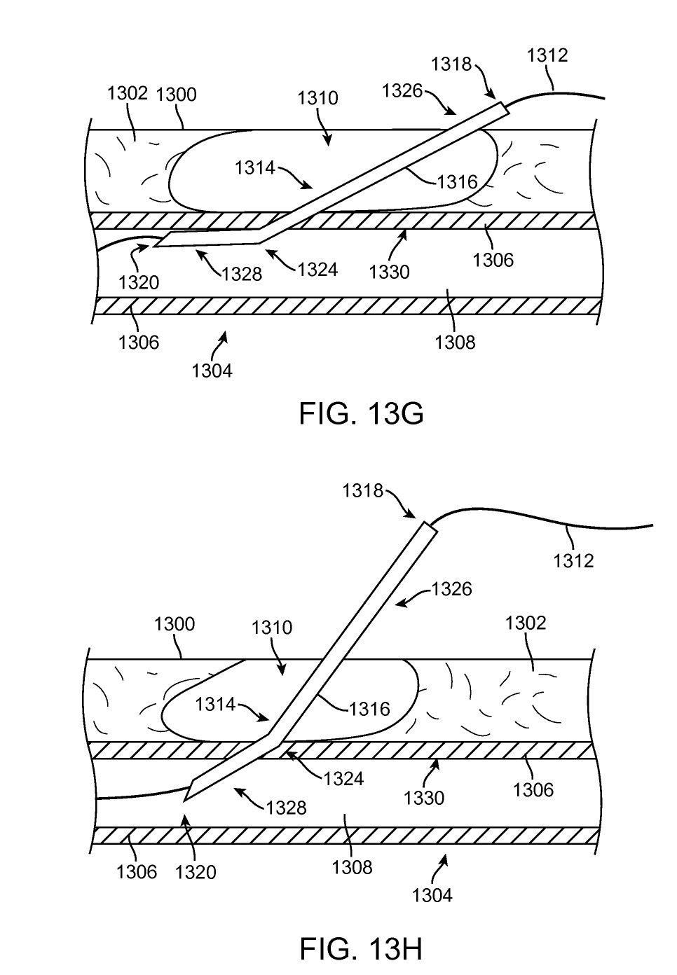

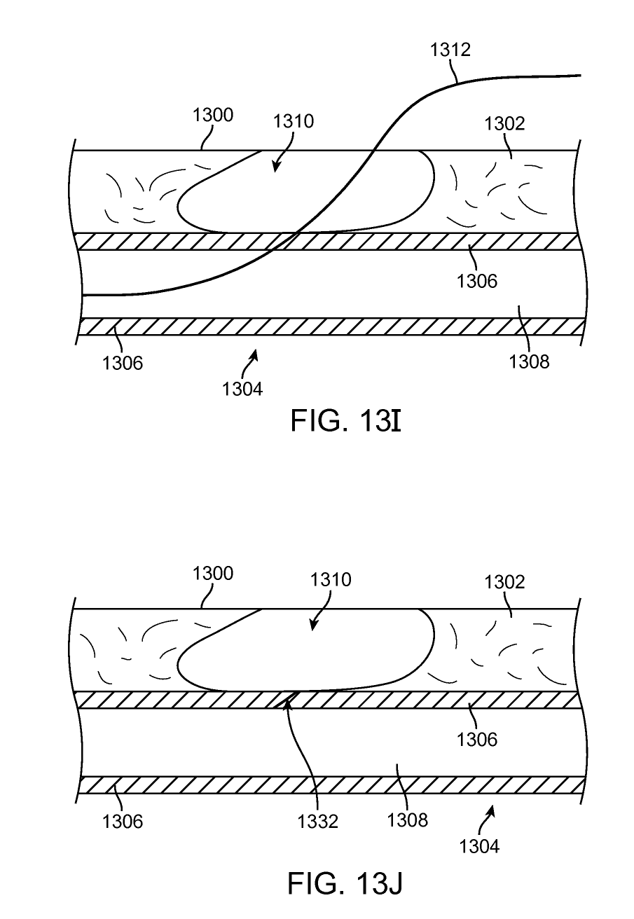

For example, FIGS. 13A-13J depict a variation of a tissue tract-forming device, as it is being used to form a tract through a vessel wall. First, FIG. 13A shows a skin surface (1300), subcutaneous tissue (1302) beneath the skin surface, and a vessel (1304) beneath the subcutaneous tissue. Vessel (1304) comprises a vessel wall (1306) defining a lumen (1308). As shown, a pocket (1310) of space has been created in subcutaneous tissue (1302), over a portion of vessel wall (1306). Pocket (1310) may be formed, for example, by dissection (e.g., blunt dissection), incision and/or dilation (e.g., using a balloon catheter passed over a guidewire placed by an initial Seldinger needle stick). Additionally, a guidewire (1312) has been routed through skin surface (1300), subcutaneous tissue (1302), and vessel wall (1306), and into lumen (1308).

FIG. 13B shows a tissue tract-forming device (1314) as it is being advanced over guidewire (1312), through skin surface (1300), and into pocket (1310). Guidewire (1312) may be any guidewire having a diameter suitable for use with device (1314). Moreover, while a guidewire is described, other variations of guide elements may be used with the devices, methods and kits described here, as appropriate. Guidewire (1312) may also have one or more expandable members (e.g., an expandable balloon, an expandable cage or flower wire formation, expandable arms, etc.) or similar such features on its distal end. In this way, the distal end of the guidewire may be used to help locate or position the device with respect to the tissue and to maintain its position for a portion of the procedure. For example, the guidewire may be advanced through the tissue, and the distal expandable feature expanded. The guidewire may then be gently pulled proximally (i.e., in the direction of the tissue). Once the expandable member abuts the tissue (as determined via tactile feedback, for example), the location of tissue has been determined and this information may be used as a guide for the rest of the procedure. Of course, these tissue location methods may not be necessary, such as when indirect (e.g., fluoroscopic guidance, ultrasound, etc.) or direct (e.g., camera, scope, etc.) visualization is employed.

Device (1314) comprises a tubular member (1316) having a proximal end (1318), a pointed, tissue-piercing distal end (1320), and a lumen (not shown) therethrough. Tubular member (1316) has a bend (1324), such that the tubular member comprises a first portion (1326) proximal to the bend, and a second portion (1328) distal to the bend.

In some variations (e.g., for vascular applications), first portion (1326) of tubular member (1316) may have a length of about 5 centimeters (1.97 inches) to about 10 centimeters (3.94 inches), such as about 7 centimeters (2.76 inches) to about 9 centimeters (3.54 inches). Alternatively or additionally, first portion (1326) may have a cross-sectional diameter of about 0.38 millimeter (0.015 inch) to about 4 millimeters (0.16 inch), such as about 1 millimeter (0.039 inch) to about 4 millimeters (0.16 inch), or about 0.38 millimeter (0.015 inch) to about 3.81 millimeters (0.15 inch). In certain variations, second portion (1328) of tubular member (1316) may have a length of about 1 centimeter (0.39 inch) to about 4 centimeters (1.57 inches), such as about 2 centimeters (0.79 inch) to about 3 centimeters (1.18 inches). Alternatively or additionally, second portion (1328) may have a cross-sectional diameter of about 1 millimeter (0.039 inch) to about 2 millimeters (0.079 inch). First portion (1326) and second portion (1328) may have at least some of the same dimensions, or may have entirely different dimensions from each other.

In some variations, first and second portions (1326) and (1328) may form an angle therebetween. The angle may be, for example, from about 90° to about 180° (e.g., from about 90° to about 175°, from about 90° to about 160°, from about 90° to about 135°, from about 90° to about 120°, from about 90° to about 100°, from about 120° to about 180°, from about 120° to about 175°, from about 135° to about 175°, from about 150° to about) 170°. In certain variations (e.g., for vascular applications), bend (1324) may have a radius of curvature of about 2 millimeters (0.079 inch) to about 19.05 millimeters (0.75 inch), such as about 2 millimeters (0.079 inch) to about 5 millimeters (0.20 inch), or about 6.35 millimeters (0.25 inch) to about 19.05 millimeters (0.75 inch). The radius of curvature of bend (1324) may be selected, for example, to permit smooth or unencumbered passage of a guidewire of a particular size (e.g., 0.014 inch, 0.018 inch, 0.035 inch, etc.). In some variations, bend (1324) may be formed by a heat-shaping process.

Tubular member (1316), and other tubular members described herein, may comprise any appropriate material or materials, such as shape-memory and/or super-elastic materials. In some cases, tubular member (1316) may comprise a nickel-titanium alloy, such as Nitinol.

As shown in FIG. 13B, distal end (1320) of tubular member (1316) has contacted vessel wall (1306). In some cases, this contact may be sensed by the operator (e.g., using one or more of the tissue-locating methods and/or components described herein).

Referring now to FIG. 13C, in preparation for advancing device (1314) through vessel wall (1306), the operator may proximally withdraw guidewire (1312), such that the guidewire no longer extends past distal end (1320) of tubular member (1316). Next, and referring also to FIGS. 13D-13F, the operator may manipulate device (1314) to advance the device through vessel wall (1306) at the desired location. The presence of pocket (1310) may allow for relatively easy manipulation of device (1314) over vessel wall (1306), so that the tip of the device may be properly positioned (e.g., at a sufficiently oblique entry angle) immediately prior to advancement through the vessel wall. As shown in FIGS. 13E and 13F, the device may be advanced through vessel wall (1306) such that second portion (1328) of tubular member (1316) is disposed within lumen (1308) of vessel (1304). In some cases, and as shown in FIG. 13F, second portion (1328) may substantially contact an inner surface (1330) of vessel wall (1306). This may, for example, provide the operator with a tactile indication that second portion (1328) has entered lumen (1308). Additionally, in some variations (not shown) in which second portion (1328) has a larger diameter than first portion (1326), the differential in diameter (and transition or change presented by bend (1324)) may provide the operator with a tactile indication that second portion (1328) is about to enter lumen (1308) of vessel (1304), is entering lumen (1308), or has entered lumen (1308). This may, for example, provide the operator with a signal that device (1314) should not be advanced any further.

Referring now to FIG. 13G, guidewire (1312) may then be distally advanced through tubular member (1316), and back into lumen (1308) of vessel (1304). Next, device (1314) may be proximally withdrawn over guidewire (1312) (FIG. 13H), leaving guidewire (1312) positioned across skin surface (1300), subcutaneous tissue (1302), and vessel wall (1306), as shown in FIG. 13I. Any suitable device or devices, such as various introducers and/or tools, may then be advanced over guidewire (1312) and into lumen (1308), so that the desired procedure or procedures may be performed. After the procedure(s) have been performed, guidewire (1312) may be proximally withdrawn, leaving behind a tract (1332) in vessel wall (1306) (FIG. 13J). In some cases, tract (1332) may be a self-sealing tissue tract, as discussed above. As described above, a self-sealing tissue tract does not need interventional devices or methods to help it seal—by definition, it seals by itself. For example, a self-sealing tissue tract does not need a plug, energy, sealants, clips, sutures, or the like to help it seal. The tract may be oblique, or may have any other appropriate configuration.

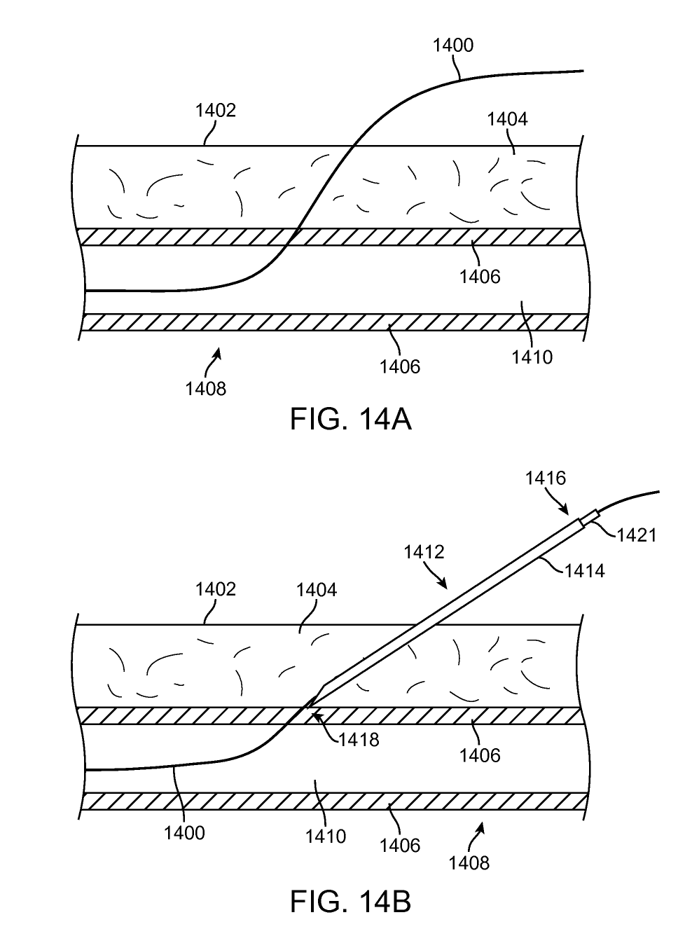

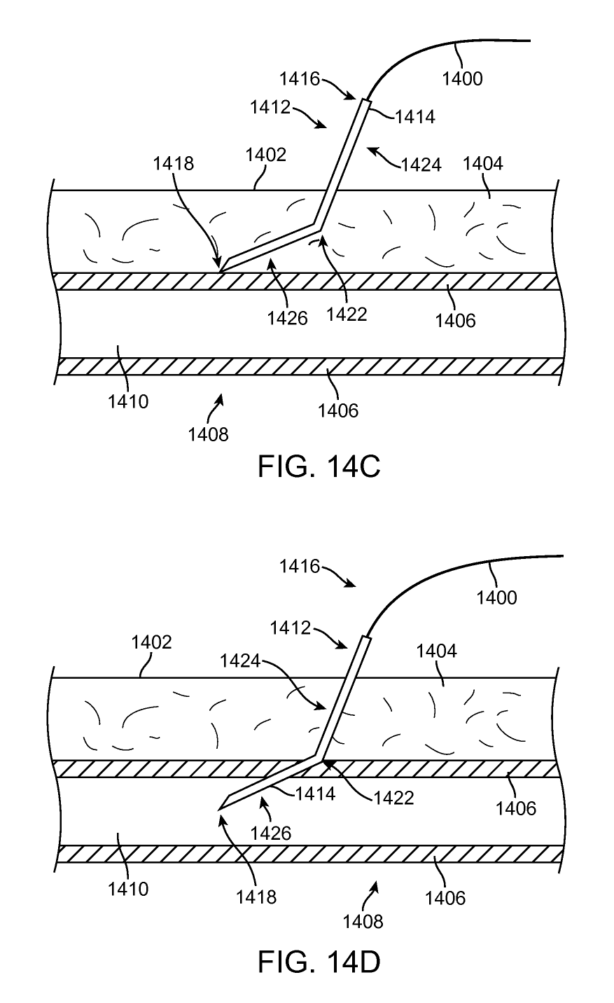

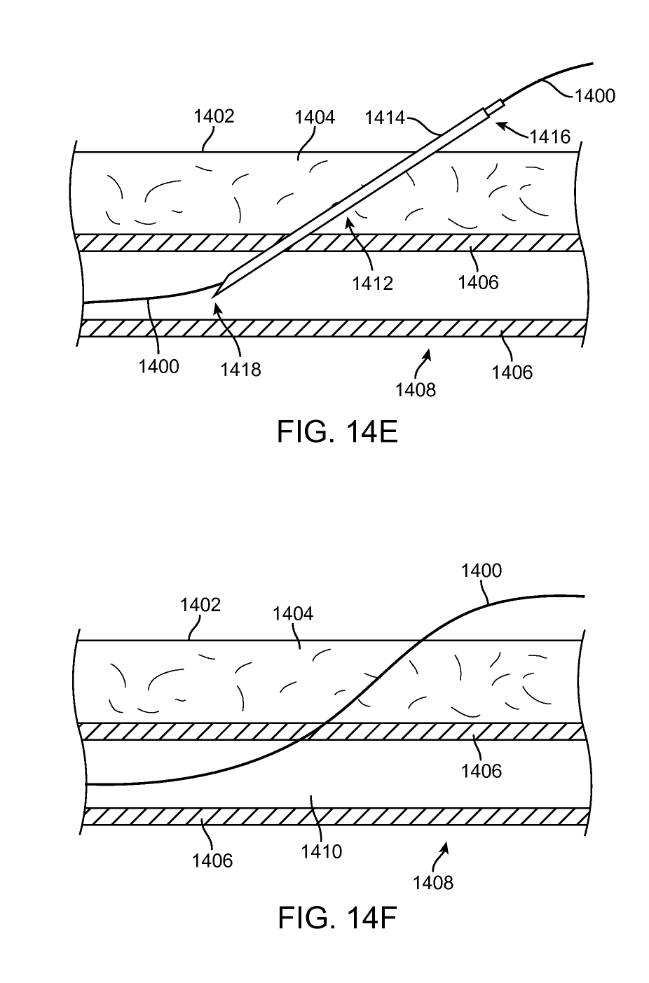

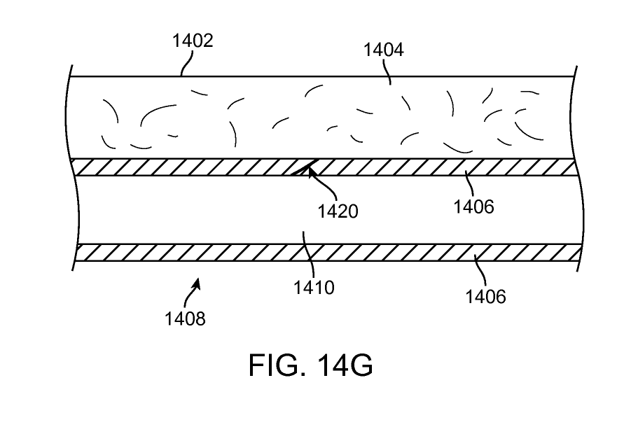

It should be noted that while device (1314) is depicted as being used with guidewire (1312), in some variations, a pocket of space such as pocket (1310) may be formed (e.g., by blunt dissection), and a device such as device (1314) may be advanced through the pocket of space and through a tissue wall without the use of a guidewire. In certain variations, a single tissue-piercing member may be used to form a single puncture through a skin surface to form a tract in tissue beneath the skin surface. In some variations, a guide element (e.g., a guidewire) may then be advanced through the tissue-piercing member (and, e.g., may be used to position one or more tools or devices at the target site). For example, the device may be positioned using any of the devices and/or methods described herein, such as those using ultrasonography, fluoroscopy, thermal sensing, visual imaging, light properties of tissue, and the like, or any other appropriate devices and/or methods, without also using a guidewire. This may be advantageous, for example, by avoiding the formation of an initial puncture for advancement of a guidewire therethrough. It may thereby allow for a tissue tract to be formed by making a single stick or puncture into the tissue. The same possibility applies for device (1412) (FIGS. 14A-14F), and for other tissue tract-forming devices, as appropriate.

It should also be noted that in some variations, device (1314) may be successfully advanced through a tissue wall, such as a vessel wall, without the need for a pocket of space, such as pocket (1310). As an example, tubular member (1316) may be capable of bluntly dissecting the subcutaneous and fatty tissue above a target vessel wall as the operator maneuvers device (1314) into position through a puncture in a skin surface.

Additionally, while not shown, in some method variations, an operator may position device (1314) such that bend (1324) is located at or just below the level of the skin surface, such as skin surface (1300). Thus, as distal end (1320) of tubular member (1316) is rotated and/or advanced to a more oblique angled position relative to vessel wall (1306), bend (1324) may pivot at the level of the puncture in skin surface (1300). As bend (1324) pivots, it may be free to move without device (1314) pushing up against the skin puncture site. As a result, any need to enlarge or extend the puncture site diameter at the skin's surface may be markedly reduced or even completely eliminated.

Of course, other variations of tissue tract-forming devices may be used, and in some cases, a tissue tract-forming device may not be bent, or may only be bent for a portion of a tissue tract-formation process. This may, for example, make it relatively easy to advance the device to the desired site for tissue tract formation.

For example, FIG. 14A shows a guidewire (1400) that has been advanced through a point of patient access, such as a skin surface (1402), through a thickness of subcutaneous tissue (1404), and through a wall (1406) of a vessel (1408), into a lumen (1410) of vessel (1408). Referring now to FIG. 14B, a tissue tract-forming device (1412), such as a needle, is being slidably advanced over guidewire (1400), toward vessel wall (1406) at a first orientation angle, defined as the angle between the tract-forming device or tip thereof and the targeted tissue wall (1406). In one embodiment, the first orientation angle may be between about 30 degrees and about 60 degrees, such as a first orientation angle of about 45 degrees. Device (1412) comprises a substantially straight tubular member (1414) having a proximal end (1416), a tissue-piercing pointed distal end (1418), and defining a lumen (not shown) therethrough sized to accommodate slidable coupling of a guidewire and/or mandrel (1421). Tubular member (1414) is maintained in its substantially straight configuration by a mandrel (1421) disposed within the lumen of tubular member (1414).

Referring now to FIG. 14C, once tubular member (1414) has been advanced to the desired location by vessel wall (1406), guidewire (1400) and mandrel (1421) may be proximally withdrawn, and tubular member (1414) may assume its natural configuration—in this case, including a bend (1422), a first portion (1424) proximal to the bend, and a second portion (1426) distal to the bend. In other words, the tubular member (1414) may comprise proximal and distal elongated portions (1424, 1426) coupled with a bending section. The bending section may be configured to assume a predetermined bent configuration when unloaded, the predetermined bent configuration being selected to place the proximal and distal portions (1424, 1426) in desired orientations relative to each other. In another embodiment, the proximal and distal portions (1424, 1426) maybe coupled with a joint, and one or more biasing members (i.e., such as a spring member or pullwire/tension member) may be coupled to the joint and configured to bias the joint to rotate to a predetermined configuration when unloaded. Either of the proximal and distal elongated portions (1424, 1426) may be substantially straight, as shown in FIGS. 14B-14DE, or bent (i.e., preconfigured to have a non-straight resting shape). In an embodiment wherein the tubular member (1414) is biased to assume a configuration such as that depicted in FIGS. 14C and 14D when unloaded, the mandrel (1421) described herein preferably has a structural stiffness to resist this biasing and maintain the other configuration, such as that depicted in FIG. 14B. In another embodiment, the mandrel may comprise structural stiffness sufficient to reconfigure the tubular member (1414) from a configuration such as those depicted in FIG. 14C and 14D back to a configuration such as that depicted in FIG. 14B when inserted back through both of the proximal and distal portions (1424, 1426). In some cases, first and second portions (1424) and (1426) may have an angle of about 90° to about 135° (e.g., about 100°) or about 120° to about 180° (e.g., about 120° to about 175°) therebetween. Alternatively or additionally, when a mandrel or other straightening feature is disposed within the lumen of tubular member (1414), first and second portions (1424) and (1426) may have an angle of about 135° to about 180° (e.g., about) 175° therebetween. As a result of controlled (i.e., by withdrawal or insertion of the mandrel in one embodiment) reorientation of the distal portion (1426), a second orientation angle, defined as the angle between the tract-forming device or tip thereof and the targeted tissue wall (1406), may describe the orientation of the distal portion (1426) as it enters the targeted tissue structure all (1406). In one embodiment, it is desirable to create a self-sealing access tract, as described in further detail herein and in the incorporated references, and a second orientation angle of between about 2 degrees and about 30 degrees, such as an angle of about 10 degrees, may be preferred.

Referring as well now to FIG. 14D, the operator may manipulate device (1412) to advance tubular member (1414) through vessel wall and into lumen (1410), so that device (1412) thereby forms a tract through the vessel wall. In one embodiment, an elongate deployment member, such as a sheath or trocar, may be movably coupled (i.e., in one embodiment via a deployment lumen defined through at least a portion of the elongate deployment member, the lumen sized to accommodate slidable coupling between the elongate deployment member and the tubular member 1414) to the tubular member (1414) and configured to be manually (i.e., with the proximal end of such elongate deployment member) manipulated by an operator to apply loads (i.e., torsional loads, axial loads) to the tubular member (1414). Once the desired tract has been formed, guidewire (1400) and mandrel (1421) may be distally advanced back through tubular member (1414) (and, in the case at least of guidewire (1400), into lumen (1410)), thereby causing tubular member (1414) to once again assume its substantially straight configuration (FIG. 14E). Tubular member (1414) may then be proximally withdrawn from the body of the subject, thereby leaving guidewire (1400) behind, positioned across skin surface (1402), subcutaneous tissue (1404), and vessel wall (1406) (FIG. 14F). Any appropriate desired procedures may then be performed, for example, as discussed above with respect to FIG. 131. Eventually, guidewire (1400) may be removed, leaving a tract (1420) within vessel wall (1406), as shown in FIG. 14G. In some cases, the tract may be self-sealing. As described above, a self-sealing tissue tract does not need interventional devices or methods to help it seal—by definition, it seals by itself. For example, a self-sealing tissue tract does not need a plug, energy, sealants, clips, sutures, or the like to help it seal.

It should noted that in some variations, a curved or angled device (such as device (1314) above) may be capable of being advanced through tissue and across a tissue wall to form a tract through the tissue wall, without having to change configurations in order to do so. For example, the curved or angled device may be capable of being advanced over a guidewire through tissue and a target tissue wall, without requiring a configurational change in order to form a tract through the tissue wall.

The methods described here may be used to locate and/or form tracts in any tissue in connection with any technique or procedure. The tissue may be any suitable tissue (e.g., tissue in which it is desirable to form a tract therethrough). For example, it may be tissue of the cardiovascular system, digestive system, respiratory system, excretory system, reproductive system, nervous system, etc. In some variations the tissue may be tissue of the cardiovascular system, such as an artery, or a heart. In other variations the tissue may be tissue that is accessed through a natural orifice (e.g., to perform natural orifice translumenal endoscopic surgery or “NOTES”), such as tissue of the reproductive system, excretory system, digestive system, or the like. Of course, it should be understood that methods of forming multiple tracts in tissue, whether through similar or different tissue, are also contemplated.

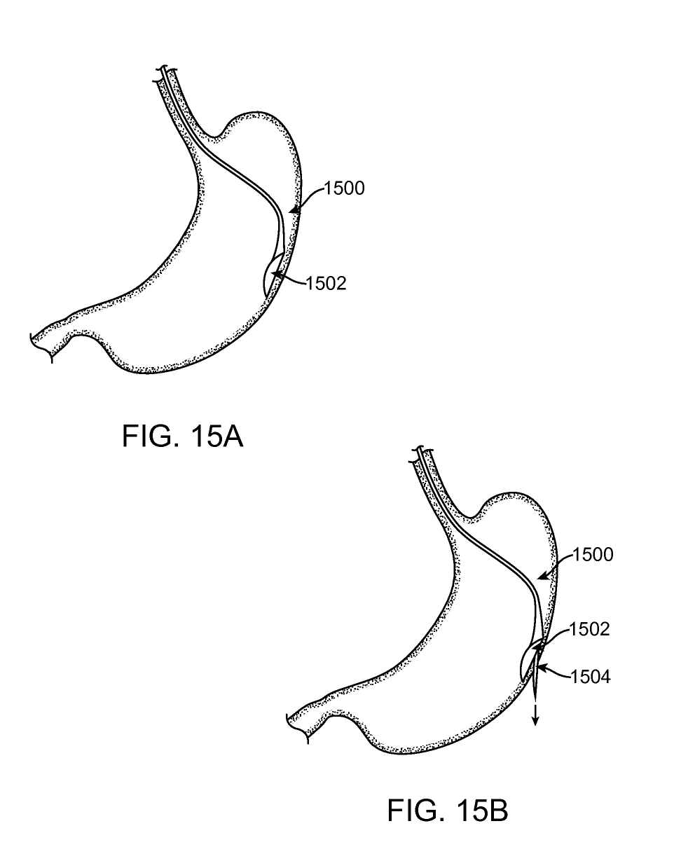

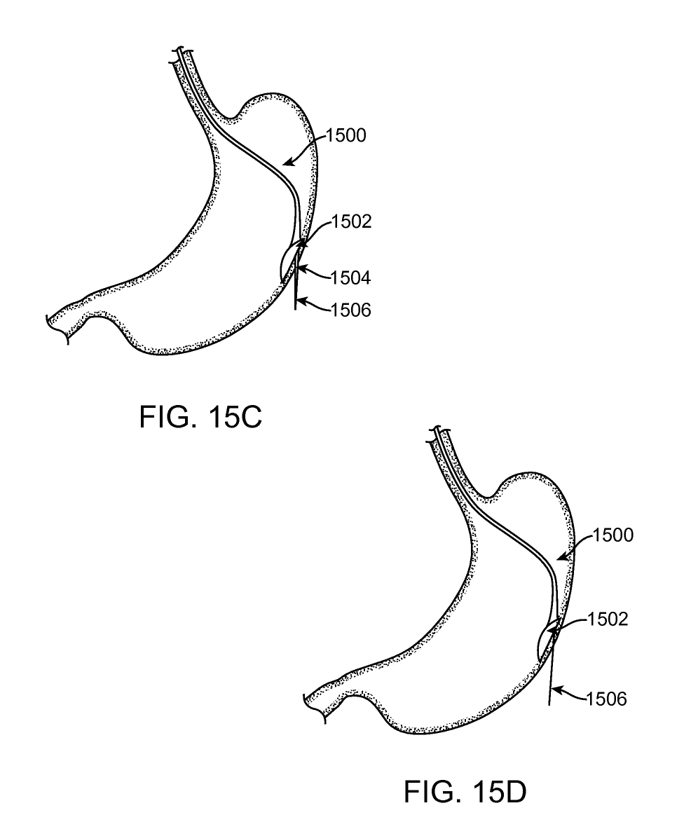

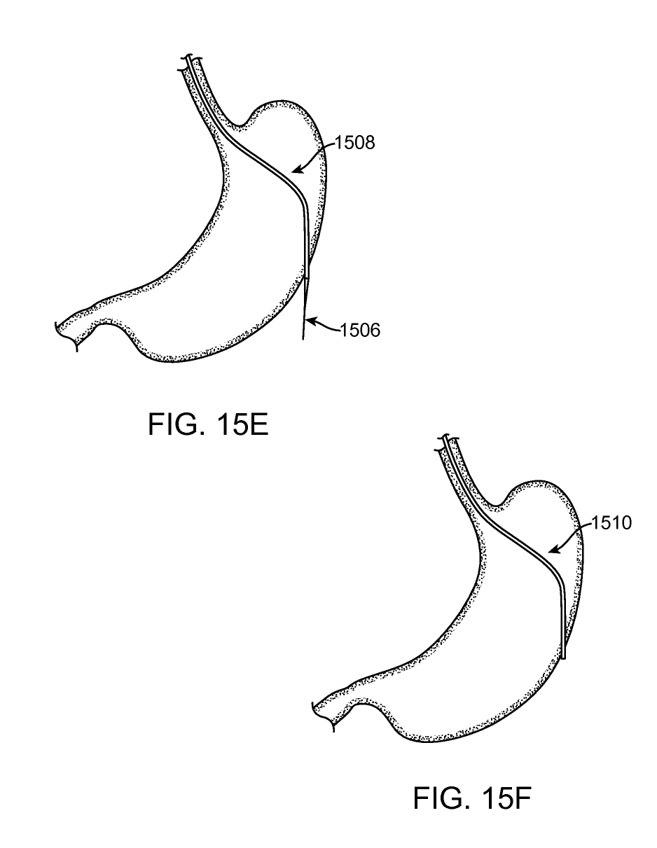

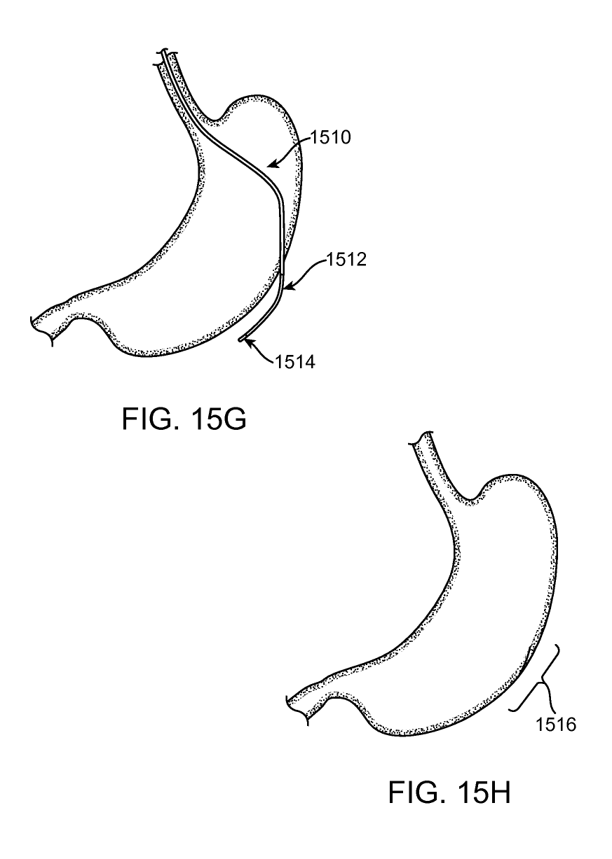

FIGS. 15A-15H depict a method of forming a tract in or through stomach tissue. It should be understood that just the distal portion of the device is shown in these figures, and that this method may be used to form tissue tracts as depicted, whether or not the device is a stand alone device, or is used with a gastroscope or advanced through some other sheathed structure (including instances where the device is back-loaded into the working channel of any type of gastroscope, endoscope, laparoscope, etc., with or without steering, visualization, illumination, etc.). Turning now to FIG. 15A, the device (1500), comprising a tissue-locating member (1502), is shown advanced adjacent to tissue, here stomach tissue. Next, a tissue-piercing member (1504) (e.g., a needle or other tissue-piercing cannula) is advanced from the device and through the tissue to form a tract in the tissue, as shown in FIG. 15C.

Once the tract has been formed, a guidewire (1506), other guide element, or the like may be advanced through the tract (e.g., by advancing through a lumen in the tissue-piercing member), as shown in FIG. 15C, and tissue-piercing member (1504) may be withdrawn, as shown in FIG. 15D. A stepped-up dilator (1508) or series of dilators (not shown) may then be advanced over guidewire (1506), as shown in FIG. 15E. In this way, for example, the cross-sectional area of the tract may be expanded or enlarged. After the tract has been expanded, an introducer (1510), which may be part of the dilator (1508), may be left in place and used as a conduit for introducing additional tools through the tract, as shown in FIG. 15F. FIG. 15G shows one illustrative method where a tool (1512) having an end effector (here, grippers (1514), although other end effectors may alternatively or additionally be used) has been advanced through introducer (1510) for use in a procedure. Any number or type of tools may be advanced through the introducer in this way. After the procedure has been performed, the tools and introducer are removed, leaving tract (1516) to seal (e.g., to self-seal). Of course, sealing may be enhanced any suitable additional mechanism (e.g., via mechanical pressure, via ultrasound, via one or more closure devices, and the like).

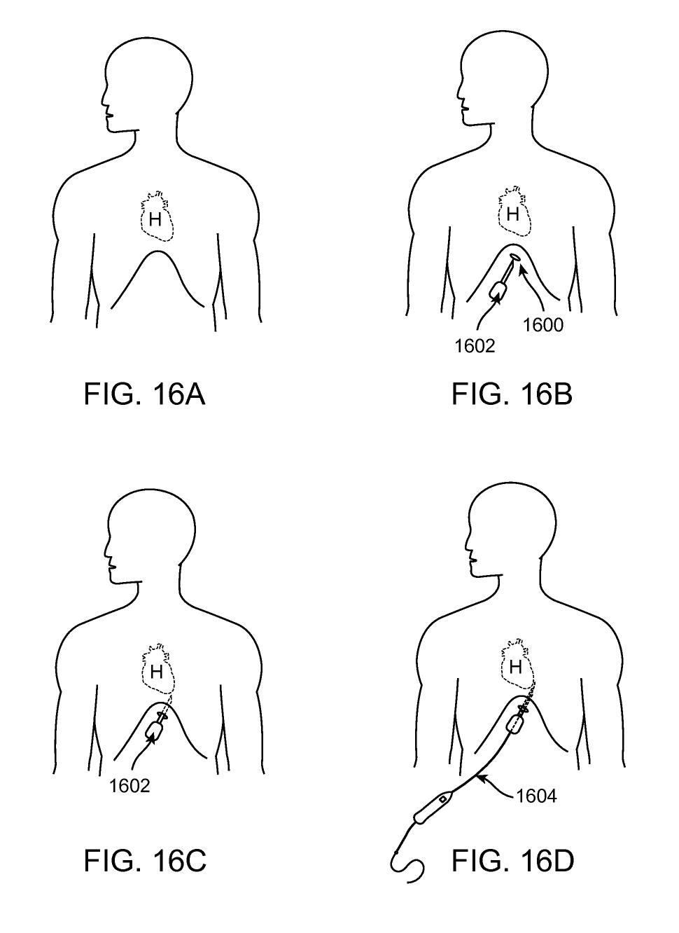

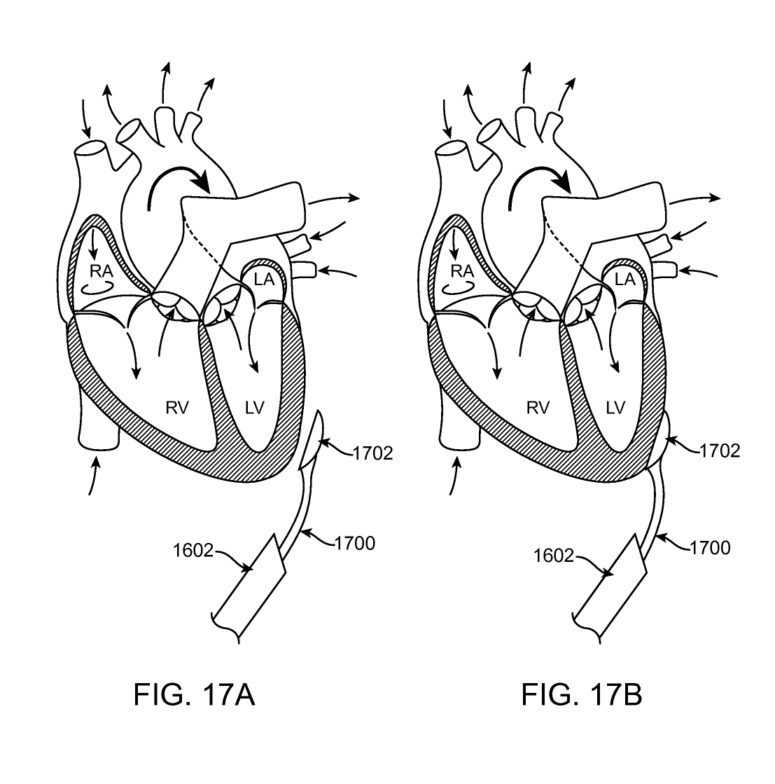

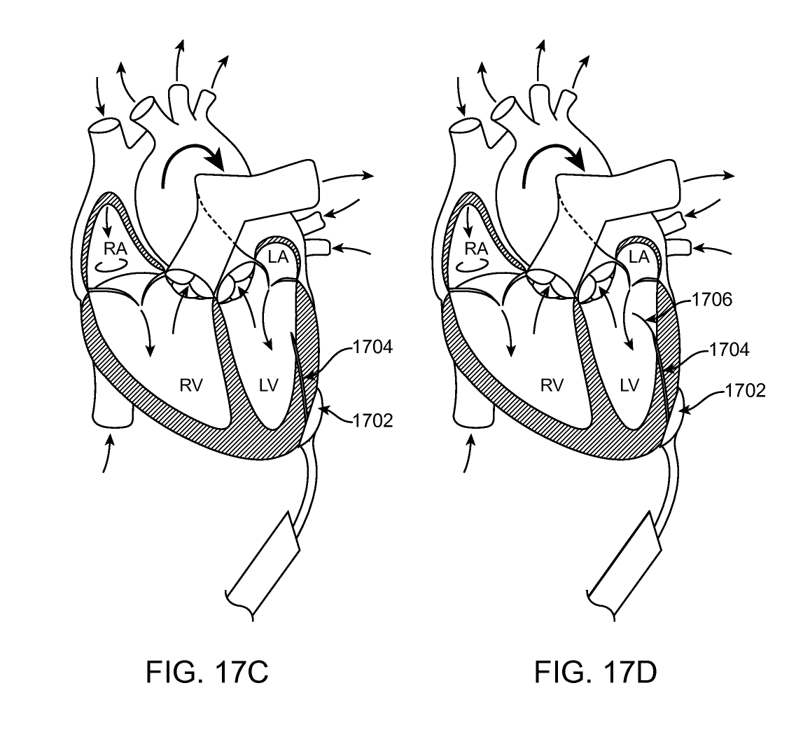

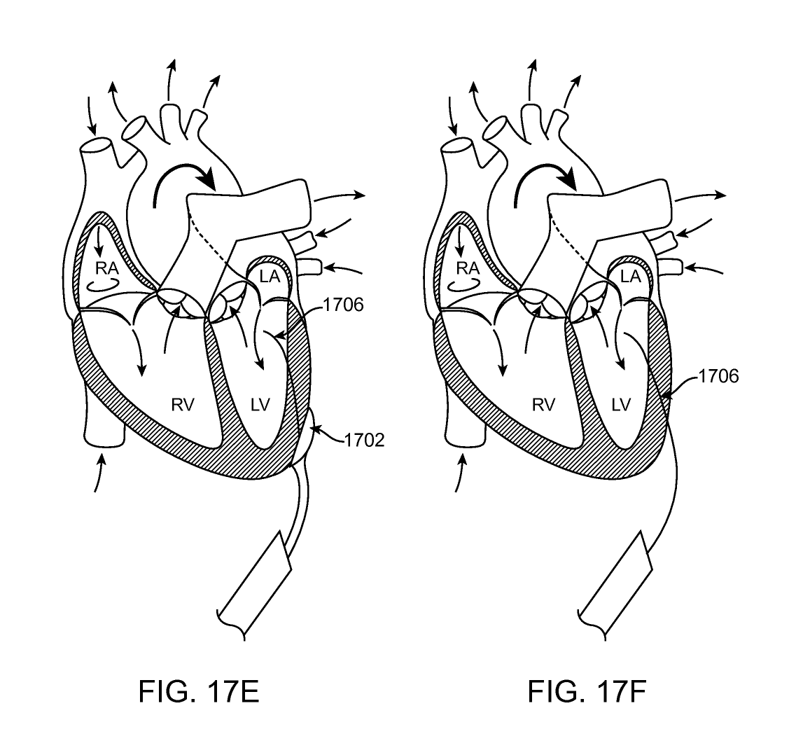

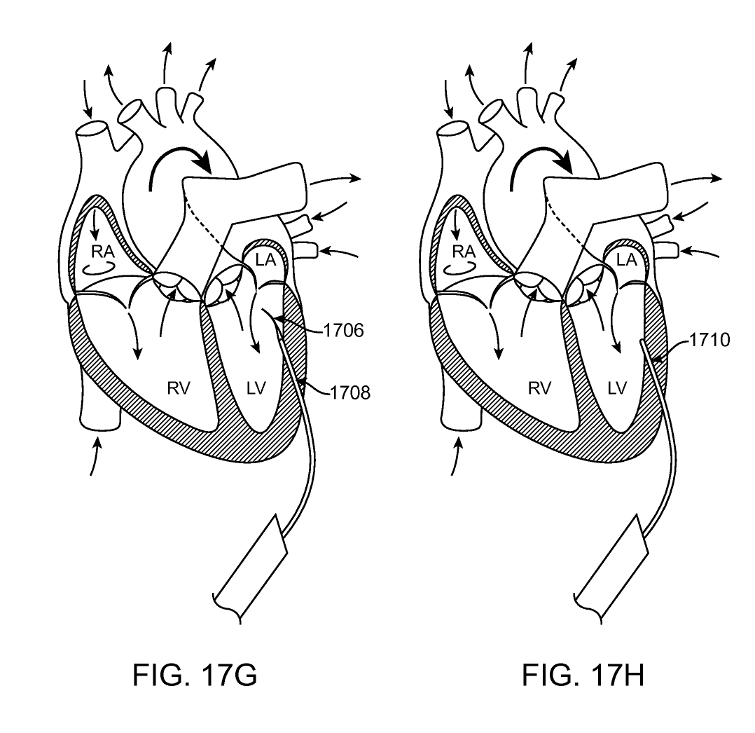

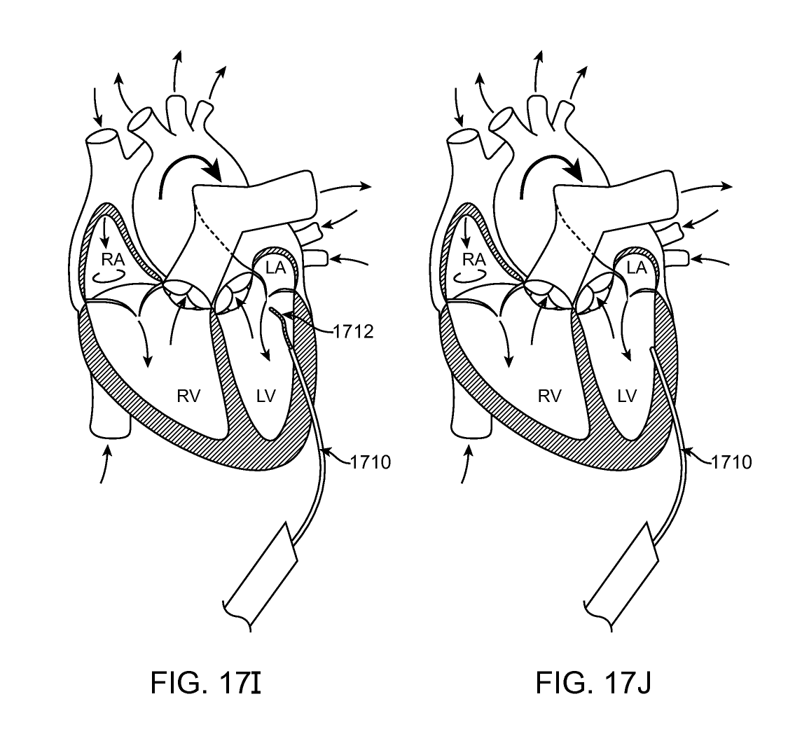

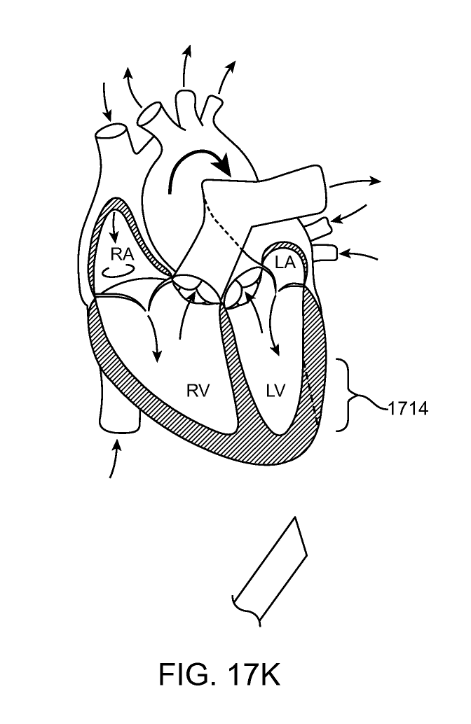

FIG. 16A-16D depict one method of advancing a device described herein into the pericardial space in order to form a tract through tissue of the heart (H). As shown in those figures, an incision (1600) may be made (e.g., sub-xyphoid, etc.) and a port (1602) placed therethrough to provide for suitable delivery or exchange of tools therethrough. Once the port (1602) has been placed, any of the devices (1604) described here, as appropriate, may be placed through the port (1602) to form a tract in or through tissue of the heart (H), as will be described in more detail with reference to FIGS. 17A-17K.

Turning to FIG. 17A, a device (1700) comprising a tissue-locating member (1702) is advanced adjacent to heart tissue. The device may be advanced adjacent to the heart tissue in any suitable fashion, such as through port (1602) described above. Tissue-locating member (1702) may be placed in contact with the heart tissue, as shown in FIG. 17B. As shown in FIG. 17C, a tissue-piercing member (1704) may then be advanced from the device (e.g., through the tissue-locating member) and through the heart tissue to form a tissue tract. A guidewire (1706) or other suitable such guide element may then be advanced through the tract, for example, by advancing through a lumen in tissue-piercing member (1704), as shown in FIG. 17D. Tissue-piercing member (1704) and device (1700) may then be removed, as shown in FIGS. 17E and 17F, respectively.

A stepped-up dilator (1708) or series of dilators (not shown) may then be advanced over guidewire (1706), as shown in FIG. 17G. In this way, for example, the cross-sectional area of the tract may be expanded or enlarged. After the tract has been expanded, an introducer (1710), which may be part of dilator (1708), may be left in place and used as a conduit for introducing additional tools through the tract, as shown in FIG. 17H. FIG. 171 shows one illustrative method where a tool (1712) has been advanced through introducer (1710) for use in a procedure. Here left ventricular access has been accomplished, and therefore, use of these methods in conjunction with repair or replacement of the aortic or mitral valve may find particular utility. Any number or type of tools may be advanced through the introducer in this way. After the procedure has been performed, the tools and introducer are removed, leaving tract (1714) to seal (e.g., to self-seal), as shown by FIGS. 17J and 17K. Of course, sealing may be enhanced by any suitable additional mechanism (e.g., via mechanical pressure, via ultrasound, via one or more closure devices, and the like).

The methods may include creating a tract that self-seals within a period of time (e.g., 15 minutes or less, 12 minutes or less, 10 minutes or less, 5 minutes or less, 3 minutes or less, 1 minute or less, etc.). As described above, a self-sealing tissue tract does not need interventional devices or methods to help it seal—by definition, it seals by itself. For example, a self-sealing tissue tract does not need a plug, energy, sealants, clips, sutures, or the like to help it seal. Of course, tracts that may otherwise self-seal after a period of time may nevertheless have sealing expedited by other mechanisms as well (e.g., application of mechanical pressure, application of suction, application of one or more sealing agents, etc.).

The methods may also comprise application of energy, delivery of one or more fluids or useful agents, delivery of one or more useful tools to a tissue site, performing a procedure, visualization, determining the location of the device with respect to the tissue, combinations thereof, and the like. The device may be rotated, repositioned, or otherwise manipulated during these methods.

Kits are also described here. In some variations, the kits may include at least one device for locating tissue, as described above. Alternatively or additionally, the kits may include at least one device for forming a tract through tissue. The kits may also comprise one or more additional tools. For example, the tools may be those that are advanced through the tract during the performance of a procedure (e.g., guide wires, scissors, grippers, ligation instruments, etc.), one or more supplemental tools for aiding in closure (e.g., an energy delivering device, a closure device, and the like), one or more tools for aiding in a procedure (e.g., gastroscope, endoscope, cameras, light sources, etc.), combinations thereof, and the like. Of course, instructions for use may also be provided with the kits.

While the devices, methods and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the disclosure, including the appended claims.

As an example, in some variations, one or more of the devices, methods and/or kits described here may be used to form one or more tracts in rotated and/or tented tissue. For example, a method may comprise positioning a device adjacent a portion of a tissue wall, rotating the portion of the tissue wall (e.g., using the device), and advancing a tissue-piercing member through the rotated tissue to form the tract. The rotating may help to position the tissue-piercing member relative to the tissue wall. The tissue may be rotated in either direction about a tissue circumference (e.g., from 0° to 360°, from 0° to 180°, from 0° to 45°, from 45° to 90°, etc.). However, the tissue need not be rotated a significant amount (e.g., the tissue may be rotated 1°, 5°, 10°, 15°, etc.) and the entire tissue thickness need not be rotated. Methods that include rotating or tenting tissue are described, for example, in U.S. patent application Ser. No. 11/873,957 (published as US 2009/0105744 A1), which is incorporated herein by reference in its entirety.

As another example, in certain variations, a method may comprise applying a vacuum to tissue and/or clamping tissue. In some variations, a method may comprise advancing a tissue-piercing member into tissue after applying a vacuum to the tissue and/or clamping the tissue. Certain variations of methods described here may also comprise clamping or otherwise isolating tissue, and positioning the tissue for relatively easy advancement of a tissue-piercing member therethrough, to form a tract in at least a portion of the tissue. Methods for applying a vacuum or suction to tissue, as well as clamping methods and other tissue-positioning or isolation methods, are described, for example, in U.S. patent application Ser. Nos. 12/507,038 (filed on Jul. 21, 2009) and Ser. No. 12/507,043 (filed on Jul. 21, 2009), both of which were previously incorporated herein by reference in their entirety.

What is claimed is:

1. A system for forming a tract in a targeted tissue structure wall located across a thickness of tissue from a point of patient access, comprising:
a. a tissue-piercing member comprising a proximal elongated portion and a distal elongated portion coupled to the proximal elongated portion, the distal portion comprising a tissue-piercing tip; and
b. a mandrel;
wherein a lumen is formed through both portions of the tissue-piercing member and configured to slidably receive the mandrel, such that when the mandrel is received by both elongated portions, the elongated portions assume a first orientation relative to each other, and when the mandrel is withdrawn proximally out of at least the distal elongated portion, the elongated portions assume a second orientation relative to each other.

2. The system of claim 1, further comprising a guidewire slideably coupled through the lumen of the tissue-piercing member and advanced from the point of patient access across at least a portion of the targeted tissue structure wall.

3. The system of claim 1, wherein the proximal and distal elongated portions of the tissue-piercing member are coupled with a bending section.

4. The system of claim 3, wherein the bending section assumes a predetermined bent configuration when unloaded.

5. The system of claim 4, wherein the predetermined bent configuration is selected to place proximal and distal elongate members in the second orientation relative to each other when coupled with the bending section and not restrained by the mandrel.

6. The system of claim 1, wherein the proximal and distal elongated portions of the tissue-piercing member are coupled with a joint.

7. The system of claim 6, further comprising a biasing member coupled to the joint and configured to bias the joint to rotate to a predetermined configuration when unloaded.

8. The system of claim 7, wherein the predetermined configuration is selected to place proximal and distal elongate members in the second orientation relative to each other when coupled with the joint and not restrained by the mandrel.

9. The system of claim 1, wherein at least one of the first and second elongated portions is substantially straight when unloaded.

10. The system of claim 1, wherein at least one of the first and second elongated portions has a bent configuration when unloaded.

11. The system of claim 1, further comprising an elongated deployment member movably coupled to the tissue-piercing member and configured to be manipulated by an operator to apply loads to the tissue-piercing member.

12. The system of claim 11, wherein the elongated deployment member defines a deployment lumen configured to accommodate slidable coupling of the tissue-piercing member with the elongated deployment member.

13. The system of claim 1, wherein the tissue-piercing member is biased to assume the second configuration when unloaded, and wherein the mandrel comprises a structural stiffness selected to maintain the tissue-piercing member in the first configuration when inserted through both the proximal and distal elongated portions.

14. The system of claim 1, wherein the tissue-piercing member is biased to assume the second configuration when unloaded, and wherein the mandrel comprises a structural stiffness selected to urge the tissue-piercing member back into the first configuration after the second configuration has been assumed, such reconfiguration being accomplished by applying insertional forces on the mandrel relative to the tissue-piercing member to insert the mandrel back through at least a portion of the lumen defined through the distal elongate portion of the tissue-piercing member.

15. The system of claim 1, wherein the tissue-piercing member is a needle.

16. The system of claim 1, wherein the tissue-piercing member comprises at least one shape-memory material.

17. The system of claim 1, wherein the tissue-piercing member comprises at least one super-elastic material.

18. The system of claim 17, wherein the super-elastic material comprises nitinol.

19. The system of claim 1, wherein an articulation angle is defined between a longitudinal axis of the proximal tissue-piercing member portion and a longitudinal axis of the distal tissue-piercing member portions, and wherein the articulation angle with the proximal and distal elongated portions in the first orientation is between about 135 degrees and about 180 degrees.

20. The system of claim 19, wherein the articulation angle with the proximal and distal elongated portions in the first orientation is about 175 degrees.

21. The system of claim 1, wherein an articulation angle is defined between a longitudinal axis of the proximal tissue-piercing member portion and a longitudinal axis of the distal tissue-piercing member portions, and wherein the articulation angle with the proximal and distal elongated portions in the second orientation is between about 90 degrees and about 135 degrees.

22. The system of claim 21, wherein the articulation angle with the proximal and distal elongated portions in the second orientation is about 100 degrees.

23. The system of claim 1, wherein the tissue-piercing member is configured to be advanced through the thickness of tissue with the tissue-piercing tip at a first orientation angle relative to the targeted tissue structure wall until the tissue-piercing tip is located adjacent the targeted tissue structure wall, after which the mandrel may be at least partially withdrawn relative to the tissue-piercing member to cause the tissue-piercing member to assume the second orientation and place the tissue-piercing tip at a second orientation angle relative to the targeted tissue structure wall that is less than the first orientation angle relative to the targeted tissue structure wall, the second orientation angle being selected to cause the tissue-piercing tip to be advanceable into the targeted tissue structure wall with a trajectory configured to leave behind a tract through the targeted tissue structure wall that is self-sealing after the tissue-piercing member has been withdrawn.

24. The system of claim 23, wherein the first orientation angle of the tissue-piercing tip relative to the targeted tissue structure wall is between about 30 degrees and about 60 degrees.

25. The system of claim 24, wherein the first orientation angle of the tissue-piercing tip relative to the targeted tissue structure wall is about 45 degrees.

26. The system of claim 23, wherein the second orientation angle of the tissue-piercing tip relative to the targeted tissue structure wall is between about 2 degrees and about 30 degrees.

27. The system of claim 26, wherein the second orientation angle of the tissue-piercing tip relative to the targeted tissue structure wall is about 10 degrees.
